SEQUENCE LISTING (13 sequences)

SEQ ID NO: 1 - protein, 595 aa, Penicillium oxalicum
RPDPKGGNLTPFIHKEGERSLQGILDNLGGRGKKTPGTAAGLFIASPNTENPNYYYTWTRDSALTAKCLIDLFEDSRAKFPIDRKYLETGIRDYVSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYQGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 2 - protein, 595 aa, synthetic construct
  REGION 1..595: note = AMG NL
RPDPKGGNLTPFIHKEGERSLQGILDNLGGRGKKTPGTAAGLFIASPNTENPNYYYTWTRDSALTAKCLIDLFEDSRAVFPIDRKYLETGIRDYVSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYQGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 3 - protein, 595 aa, synthetic construct
  REGION 1..595: note = AMG anPAV498
RNDSKGGNLTFFIHKEGERSLQGILDNLGGRGKKTPGTAAGLFIASPNTENPNYYYTWTRDSALAAKCLIDLFEDSRAVFPIDRKYLETGIRDYVSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYFGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 4 - protein, 595 aa, synthetic construct
  REGION 1..595: note = AMG JPO-001
ANDSKGGNLTFFIHKEGERSLQGILDNLGGRGKKTPGTAAGLFIASPNTENPNYYYTWTRDSALAAKCLIDLFEDSRAVFPIDRKYLETGIRDYVSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYFGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 5 - protein, 595 aa, synthetic construct
  REGION 1..595: note = AMG JPO-124
ANDSKSTNLTFFIHKEGERSLQGILDNLGGFGKYTPGTAAGLFIASPNTENPNYYYTWTRDSALAAKCLIDLFEDSRAVFPIDRKYLETGIRDYVSSQAILQNVSNPSGTLKDGSGLGEPKFEIDLNPFSGPWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYFGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRNASVPPSWGAKSANKVPTTCSASPVVGTYKAPTATFPSKTKCVPAKDIVPITFYLIENTYYGENVFMSGNITALGNWDAKKGFPLTANLYTQDQNLWFASVEFIPAGTPFEYKYYKVEPNGTIVWEKGPNRVFVAPTGCPVQPHSNDVWRS

SEQ ID NO: 6 - protein, 595 aa, synthetic construct
  REGION 1..595: note = AMG JPO-172
ANDSKSTNLTFFIHKEGERSLQGILDNLGGFGKYTPGTAAGLFIASPNTRNPNYYYTWTRDSALAAKCLIDLFEDSRAVFPIDRKYLETGIRDYVSSQAILQNVSNPSGTLKDGSGLGEPKFEIDLNPFSGPWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYFGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRNASVPPSWGAKSANKVPTTCSASPVVGTYKAPTATFPSKPKCVPAKDIVPITFYLIANTYYGENVFMSGNITALGNWDAKKGFPLTANLYTQDQPLWFASVEFIPAGTPFEYKYYKVEPNGTIVWEKGPNRVFVAPTGCPVQPHSNDVWRS

SEQ ID NO: 7 - protein, 593 aa, Penicillium miczynskii
NPTSKDAKMASYISKEGQRSIVGITENLGGKGSKTPGTAAGLFIASPNMANPNYYYTWTRDSALTFKCLIDLFETSDQDYISRKELETDIRNYVSSQAVLQNVSNPSGTLKDGSGLGEPKFEIDLNPFSGSWGRPQRDGPALRATAMITYADWLISHGSKSEAANIMWPIIANDLAYVGQYWNNTGFDLWEEVDGSSFFTLAVQHRSLVQGASLAKKLGKSCPACVSQAPQILCFLQSFWNGNYITANINLDTSRSGIDLNSILGSIHTFDPEASCDDSTFQPCSARALANHKVYVDAFRSIYKVNAGLSNGSAANVGRYPEDVYQGGNPWYLATLASAELLYDALYQWSKIGKLDVTKTSLAFFKDFDMAVKTGTYSAHSSTYKSLTSAIRTYADDFIGLVQDYTPSNGSLAEQYDRNTGIPLSANDLTWSYASFITAIQRRASVVPASWGEKSANVVPTICSASPVTGTYQAAPSVFPTTTGCVPATSIVPITFYLTETTFYGENVYMTGNISALGNWDTNNGFPLTANLYTDSDHLWFASVELVPAGTPFEYKYYKVEPNGTVIWENGENRVYVAPTGCPIQPSQTDVWR

SEQ ID NO: 8 - protein, 590 aa, Penicillium russellii
SKDGNLASYIAKEGQRSIVGITENLGGKGSKTPGTAAGLFIASPNMANPNYYYTWTRDSALTFKCLIDLFETSDQDYISRKELETDIRNYVSSQAVLQNVSNPSGTLKDGSGLGEPKFEIDLNPFSGSWGRPQRDGPALRATAMITYADWLVSHGQKSEATNIMWPIIANDLAYVGQYWNKTGFDLWEEVDGSSFYTLAVQHRALVQGASLAKKLGKSCTACVSQAPQILCFLQSFWNGNYITANINLDTSRSGIDLNSILGSIHTFDPEASCDDSTFQPCSARALANHKVYVDAFRSIYGVNAGLSNGTAANVGRYPEDVYQGGNPWYLATLAAAELLYDALYQWNQIGKLDVTKTSLAFFKDFDAAVKTGTYSAHSSAYRTLTSAIRTYADDFISLVQHYTPSNGSLAEQYDRDTGIPLSANDLTWSYASFITAIERRASVVPASWGEKSANVVPTTCSASPVTGTYVAATSVFPTTTGCVPATSIVPITFYLTESTFYGENVYMTGNISALGNWDTSSGFPLTANLYTDSDHLWFASVELVPAGTPFEYKYYKVEPNGTVIWENGENRVYVAPTGCPIQPSQTDIWR

SEQ ID NO: 9 - protein, 594 aa, Penicillium glabrum
NPFNSLDSFILKEGARSYQGIIDNLGNKGVKAPGTAAGLFVASPNTANPDYFYTWTRDSALTFKCLIDLFDGGSTEFGLKNSELETDIRNYVSSQAVLQNVSNPSGTLEDGTGLGEPKFEVDLNPFTGSWGRPQRDGPALRATALITYTNYLLSQGQKSEAVNIMWPIISNDLAYVGQYWNDTGFDLWEETDGSSFFTLAVQHRALVQGATLAQKLGKSCAACSSQAPQILCFLQSFWNGKYITANINLDTSRTGIDANTLLGSIHTFDPEAACDDSTFQPCSARALANHKVYVDAFRSIYKINSGIAEGSPANVGRYPEDVYQGGNPWYLTTLASAELLYDALYQWNKIGGLDVTETSLAFFKDFHSSVKTGSYSAHSQTYKTLTSAIRTYADGFVGLVQKYTPANGSLAEQYNRNTSVPLSANDLTWSFASFLTAIQRRESIVPGSWGEKSANTVPTTCSASPVTGTYKAATSTFPTSTAGCVPATDVVPITFYLIENTYYGENVYMTGNISALGNWDTSDGLALDAGLYTETDNLWFGTLELVTAGTPFEYKYYKIEPNGTVTIWESGDNRVSVVPTGCPIQPSLHDVWRS

SEQ ID NO: 10 - protein, 591 aa, Rasamsonia emersonii
ATGSLDSFLATETPIALQGVLNNIGPNGADVAGASAGIVVASPSRSDPNYFYSWTRDAALTAKYLVDAFIAGNKDLEQTIQQYISAQAKVQTISNPSGDLSTGGLGEPKFNVNETAFTGPWGRPQRDGPALRATALIAYANYLIDNGEASTADEIIWPIVQNDLSYITQYWNSSTFDLWEEVEGSSFFTTAVQHRALVEGNALATRLNHTCSNCVSQAPQVLCFLQSYWTGSYVLANFGGSGRSGKDVNSILGSIHTFDPAGGCDDSTFQPCSARALANHKVVTDSFRSIYAINSGIAEGSAVAVGRYPEDVYQGGNPWYLATAAAAEQLYDAIYQWKKIGSISITDVSLPFFQDIYPSAAVGTYNSGSTTFNDIISAVQTYGDGYLSIVEKYTPSDGSLTEQFSRTDGTPLSASALTWSYASLLTASARRQSVVPASWGESSASSVPAVCSATSATGPYSTATNTVWPSSGSGSSTTTSSAPCTTPTSVAVTFDEIVSTSYGETIYLAGSIPELGNWSTASAIPLRADAYTNSNPLWYVTVNLPPGTSFEYKFFKNQTDGTIVWEDDPNRSYTVPAYCGQTTAILDDSWQ

SEQ ID NO: 11 - protein, 583 aa, Rhizomucor pusillus
ATSDDWKGKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFDDASLYHPKCTIDYNNQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKDGLPAIFTSATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 12 - protein, 513 aa, synthetic construct
  REGION 1..513: note = Thermostable variant alpha amylase
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGKAWDWEVDTEFGNYDYLMYADLDMDHPEVVTELKNWGKWYVNTTNIDGFRLDAVKHIKFSFFPDWLSYVRSQTGKPLFTVGEYWSYDINKLHNYITKTDGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGGTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 13 - protein, 274 aa, Bacillus licheniformis
AQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATLNGTSMASPHVAGAAALILSKHPNLSASQVRNRLSSTATYLGSSFYYGKGLINVEAAAQ

(19) United States
(12) Patent Application Publication
(10) Pub. No.: US 2025/0270485 A1
(43) Pub. Date: Aug. 28, 2025

(54) BREWING WITH THERMOSTABLE AMG VARIANTS

(71) Applicant: Novozymes A/S, Bagsvaerd (DK)

(72) Inventors: Alexander MAUCH, Ishoj (DK); Carsten ANDERSEN, Vaerloese (DK); Lucia MARRI, Frederiksberg (DK)

(73) Assignee: Novozymes A/S, Bagsvaerd (DK)

(21) Appl. No.: 18/859,642

(22) PCT Filed: Nov. 30, 2022
(86) PCT No.: PCT/EP2022/083872

§ 371 Date: Oct. 24, 2024

(30) Foreign Application Priority Data
May 4, 2022 (EP) 22171528.7

(51) Int. Cl. C12C 7/04; C12N 9/34

(52) U.S. Cl. CPC C12C 7/04; C12N 9/2428; C12Y 302/01003

(57) ABSTRACT

The invention relates to methods of producing a brewer's wort comprising adding to a mash a mature thermostable variant of a parent glucoamylase at least 70% identical to SEQ ID NO:1, SEQ ID NO:6, SEQ ID NO:7, SEQ ID NO:8 or SEQ ID NO:10 before, during or after 5 liquefaction of starch, as well as mashing or brewing compositions comprising said mature thermostable variant of a parent glucoamylase.

### REFERENCE TO SEQUENCE LISTING

This application contains a Sequence Listing in computer readable form, which is incorporated herein by reference.

### FIELD OF THE INVENTION

The invention relates to methods for producing brewers wort, said methods comprising adding a mature thermostable variant of a parent glucoamylase at least 70% identical to SEQ ID NO: 1, SEQ ID NO: 6, SEQ ID NO: 7, SEQ ID NO: 8 or SEQ ID NO: 10 to a mash; as well as mashing or brewing compositions comprising said variant.

### BACKGROUND OF THE INVENTION

Grain starches are composed of glucose polymers in which the glucose residues are linked by either alpha-1,4 or alpha-1,6 bonds. The extraction of grain constitutes in brewing is called mashing. During this step, water is adsorbed by the starch granules and its intermolecular bonds of starch are progressively disrupted until the starch is irreversibly dissolved in the water. The temperature needed to initiate this process (called swelling) is depending on the type of starch. Some cereal grain starches like barley or wheat starch start swelling at 58-65° C., whereas others like corn, rice, sorghum starches start swelling between 60-85° C. In the context of brewing with cereals possessing high pasting temperatures, typically a parallel process, the so-called liquefaction or cereal cooking is applied, where the solubilization and liquefaction (typically achieved with thermo-stable alpha-amylases) of its starch is secured by exposing the starch to temperatures of 85-100° C. for a defined time. After this step the liquefied starch is adapted (by cooling or mixing with cold malt mash) to temperatures at which saccharification enzymes can hydrolyze the present non-fermentable dextrin into fermentable sugars. The major fermentable sugars are glucose, maltose, and maltotriose while traces of sucrose and fructose are also present.

Wort produced without supplementation of exogenous enzymes, typically consists of 65-80% fermentable sugars, and dextrins ranging from 20-35%. The degree of fermentable sugars is depending on the amylolytic activity of the malt-endogenous amylases and the mashing regime applied. Due to the natural limits of the malt-endogenous amylolytic activity (i.e. β-amylase), there will be always residual non-fermentable dextrins which cannot be converted by the brewing yeast into ethanol.

Breweries targeting an increase in ethanol yields per unit raw material without significantly changing sensorial perception of the final beer, can overcome this limit using commercial pullulanases, amyloglucosidases, alpha-amylases, beta-amylases, maltogenic amylases and combination of these in the saccharification step of liquefied starch. Furthermore, reduced calorie beers and beers low in residual carbohydrates are very popular in the U.S. beer market and recently got a lot of global attention. Such beers are obtained by the same principle of using commercial saccharification enzymes but targeting maximal possible hydrolysis of dextrins.

Another field of application for commercial saccharification enzymes, is the production of wort using 100% un-malted raw materials like barley, wheat, sorghum, corn, rice, cassava etc. Since such raw materials are lacking or only contain insufficient endogenous amylolytic activity for brewing purposes, the use of commercial amylolytic enzymes is mandatory. As for mashing with barley malt, the mashing regime design needs to assure that grain starch is completely gelatinized and liquefied and subsequent saccharification has proceeded to the degree, that fermentable sugars in wort can deliver the targeted real degree of fermentation by yeast.

For raw materials displaying high gelatinization temperatures (e.g. rice, corn, sorghum), the mashing regime typically includes a liquefaction step at temperatures of 85-100° C. prior to saccharification at lower temperatures of 62-70° C. Such processes are typically more time demanding and potentially require additional equipment.

To simplify and shorten the mashing process without affecting process efficiency, improved and more thermostable amylolytic enzymes are needed.

U.S. Pat. No. 3,379,534 describes preparation of a low dextrin beer by using amyloglucosidase.

U.S. Pat. No. 4,536,477 describes a thermostable glucoamylase especially useful for preparation of glucose containing syrups from starch.

WO 2009/075682 describes the use of a certain pullulanase to produce a brewers wort where mashing is achieved using a smaller amount of enzyme protein.

Matthews et al., 2001, Journal of Institute of brewing 107(3) pp 185-194 discloses preparation of a low carbohydrate beer by mashing at high temperature with glucoamylase, which is derived from Aspergillus niger.

WO 2012/140075 (Novozymes A/S, Denmark) discloses methods for production of brewers wort by adding a certain glucoamylase.

### SUMMARY OF THE INVENTION

The inventors found that thermostable variants of certain glucoamylases showed greatly improved performance in mashing i.e. brewing. Another improved performance of the thermostable variants was that they increased the sweetness or sweet taste of the product, which allowed a reduction in the amount of added sugar in traditional recipes.

Accordingly in a first aspect, the invention relates to methods of producing a brewer's wort comprising adding to a mash a mature thermostable variant of a parent glucoamylase at least 70% identical to SEQ ID NO:1, SEQ ID NO:6, SEQ ID NO:7, SEQ ID NO:8 or SEQ ID NO: 10 before, during or after liquefaction of starch.

A second aspect of the invention relates to mashing or brewing compositions comprising a mature thermostable variant of a parent glucoamylase as defined in the first aspect; preferably also comprising one or more additional enzyme selected from the group consisting of a alpha amylase, maltogenic amylase, raw-starch degrading alpha amylase, beta amylase, aminopeptidase, carboxypeptidase, catalase, cellobiose oxidase, cellulase, chitinase, cutinase, cyclodextrin glycosyltransferase, esterase, deoxyribonuclease, glucan 1,4-alpha-maltotetrahydrolase, glucanase, beta glucanase, galactanase, alpha-galactosidase, beta-galactosidase, cellobiose oxidase, glucose oxidase, alpha-glucosidase, beta-glucosidase, haloperoxidase, hemicellulytic enzyme, invertase, laccase, lipase, mannanase, mannosidase, oxidase, pectinolytic enzymes, peptidoglutaminase, peroxidase, phospholipase, phytase, polyphenoloxidase, protease, pullulanase, ribonuclease, transglutaminase, and xylanase.

### FIGURES

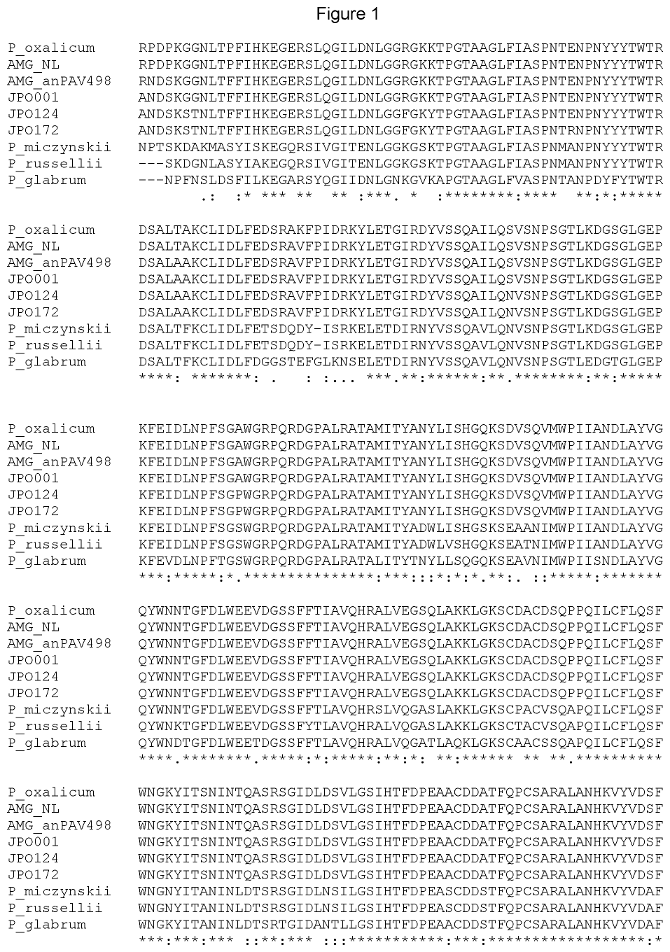

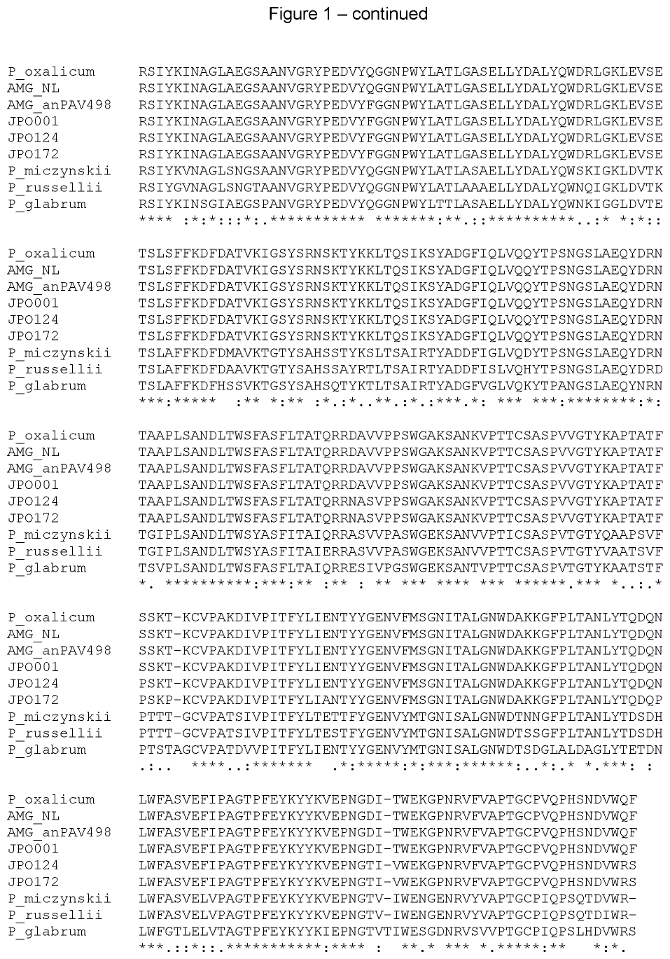

FIG. 1 shows a multiple alignment of the amino acid sequences of the mature proteins of:
Wildtype AMG from Penicillium oxalicum (PoAMG) of SEQ ID NO:1
PoAMG variant denoted ‘AMG NL’ of SEQ ID NO:2
PoAMG variant denoted ‘AMG anPAV498’ of SEQ ID NO:3
PoAMG variant denoted ‘AMG JPO001’ of SEQ ID NO:4
PoAMG variant denoted ‘AMG JPO124’ of SEQ ID NO:5
PoAMG variant denoted ‘AMG JPO-172’ of SEQ ID NO:6
Wildtype AMG from Penicillium miczynskii (PoAMG) of SEQ ID NO:7
Wildtype AMG from Penicillium russellii (PoAMG) of SEQ ID NO:8
Wildtype AMG from Penicillium glabrum (PoAMG) of SEQ ID NO:9

### DETAILED DESCRIPTION OF THE INVENTION

### Definitions

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 5.0.0 or later. The parameters used are gap open penalty of 10, gap extension penalty of 0.5, and the EBLOSUM62 (EMBOSS version of BLOSUM62) substitution matrix. The output of Needle labelled “longest identity” (obtained using the −no brief option) is used as the percent identity and is calculated as follows:

$$( Identical Residues \times 100 ) / ( Length of Alignment - Total Number of Gaps in Alignment )$$

Variant: The term “variant” means a polypeptide comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding one or more amino acids adjacent to and immediately following the amino acid occupying a position. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope, or a binding domain. Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Thermostability improvement: The thermostability improvement (Td) in ° C. is a measure of how much the variants have improved in thermostability over their parent glucoamylase under the same conditions, determined as exemplified herein.

As used herein the term “grist” is understood as the starch or sugar containing material that is the basis for beer production, e.g. the barley malt and the adjunct. Generally, the grist does not contain any added water.

The term “malt” is understood as any malted cereal grain, in particular barley.

The term “adjunct” is understood as the part of the grist which is not barley malt. The adjunct may be any starch rich plant material e.g. unmalted grain, such as, but not limited to, barley, corn, rice, sorghum, and wheat and also includes readily fermentable sugar and/or syrup. The starch of some of the adjuncts has a relatively low gelatinization temperature which enable them to be mashed in together with the malt, whereas other adjuncts such as rice, corn and sorghum has a higher gelatinization temperature, such adjuncts are typically separately cooked and liquefied with an alpha-amylase before they are added to the mash

The term “mash” is understood as a starch containing slurry comprising crushed barley malt, crushed unmalted grain, other starch containing material, or a combination hereof, steeped in water to make wort.

The term “wort” is understood as the unfermented liquor run-off following extracting the grist during mashing.

The term “spent grains” is understood as the drained solids remaining when the grist has been extracted and the wort separated.

The term “beer” is here understood as fermented wort, i.e. an alcoholic beverage brewed from barley malt, optionally adjunct and hops. The term “beer” as used herein is intended to cover at least beer prepared from mashes prepared from unmalted cereals as well as all mashes prepared from malted cereals, and all mashes prepared from a mixture of malted and unmalted cereals. The term “beer” also covers beers prepared with adjuncts, and beers with all possible alcohol contents.

The term “starch gelatinization” is understood as the irreversible order-disorder transition that starch undergoes when heated in the presence of water. Differential Scanning calorimetry (DSC) is one technique that can be employed to study the gradual process of starch gelatinization describing the onset and peak temperature (T. & Tp) of starch gelatinization. The term “onset gelatinization temperature (To)” is understood as the temperature at which the gelatinization begins. The term “peak gelatinization temperature (Tp)” is understood as the temperature at endotherm peak. The term “conclusion gelatinization temperature (Tc)” is understood as the temperature at which the gelatinization has terminated.

### Wort Production

The first aspect of the invention relates to methods of producing a brewer's wort comprising adding to a mash a mature thermostable variant of a parent glucoamylase at least 70% identical to SEQ ID NO:1, SEQ ID NO:6, SEQ ID NO:7, SEQ ID NO:8 or SEQ ID NO: 10 before, during or after liquefaction of starch.

Preferably, the method of the first aspect comprises adding to a mash a mature thermostable variant of a parent glucoamylase at least 71%, e.g. at least 72%, e.g. at least 73%, e.g. at least 74%, e.g. at least 75%, e.g. at least 76%, e.g. at least 77%, e.g. at least 78%, e.g. at least 79%, e.g., at least 80%, e.g. at least 81%, e.g. at least 82%, e.g. at least 83%, e.g. at least 84%, e.g., at least 85%, e.g. at least 86%, e.g. at least 87%, e.g. at least 88%, e.g. at least 89%, e.g., at least 90%, e.g., at least 91%, e.g., at least 92%, e.g., at least 93%, e.g., at least 94%, e.g., at least 95%, e.g. at least 96%, e.g., at least 97%, e.g., at least 98%, e.g., at least 99% identical to SEQ ID NO:1, SEQ ID NO:6, SEQ ID NO:7, SEQ ID NO:8 or SEQ ID NO: 10 before, during or after liquefaction of starch.

The mashing process generally applies a controlled stepwise increase in temperature, where each step favors one enzymatic action over the other, eventually degrading proteins, cell walls and starch. Mashing temperature profiles are generally known in the art. In the present invention a saccharification (starch degradation) step in the mashing process is preferably performed between 60° C. and 66° C., more preferably between 61° C. and 65° C., even more preferably between 62° C. and 64° C., and most preferably between 63° C. and 64° C. In a particular embodiment of the present invention the saccharification temperature is 64° C.

In one aspect, the mashing process of the present invention includes but not limited to a mashing-off step. In one aspect, the mashing-off step includes but not limited to incubation of the mash at a temperature of at least 65° C. for at least 20 minutes. In one aspect, the mashing-off step comprises incubation of the mash at a temperature of at least 65° C., e.g., at least 66° C., at least 67° C., at least 68° C., at least 69° C., at least 70° C., at least 71° C., at least 72° C., at least 73° C., at least 74° C. or at least 75° C., at least 76° C., at least 77° C., at least 78° C., at least 79° C., at least 80° C., at least 81° C., at least 82° C., at least 83° C., at least 84° C. or at least 85° C. for at least 20 minutes e.g., at least 25 minutes, at least 30 minutes, at least 35 minutes, at least 40 minutes, at least 45 minutes, at least 50 minutes, at least 55 minutes, at least 60 minutes, at least 65 minutes, at least 70 minutes, at least 75 minutes, at least 80 minutes, at least 85 minutes, at least 90 minutes, at least 95 minutes, at least 100 minutes, at least 105 minutes, at least 110 minutes, at least 115 minutes, at least 120 minutes, at least 125 minutes, at least 130 minutes, at least 135 minutes, at least 140 minutes, at least 145 minutes such as at least 150 minutes. In a particular embodiment the mashing-off is done at 75° C. for 120 minutes.

In one embodiment, the invention relates to a method of producing a brewer's wort comprising adding to a mash, mature thermostable variant of a parent glucoamylase that is active during mashing conditions at temperatures of at least 65° C. In another aspect, the mature thermostable variant of a parent glucoamylase is active during mashing conditions at at least 68° C., e.g., 70° C., e.g., 72° C., for e.g. 75° C., for e.g. 76° C., e.g., 77° C., e.g., 78° C., e.g., 79° C., e.g., 80° C.

Preferably, the mature thermostable variant of the parent glucoamylase is active during lautering, mashing and/or mash filtration.

In accordance with one aspect the mash is obtainable by grounding a grist comprising malt and/or adjunct. Water may preferably be added to the grist, be preheated in order for the mash to attain the desired mash temperature at the moment of mash forming. If the temperature of the formed mash is below the desired mashing temperature, additional heat is preferably supplied in order to attain the desired process temperature. Preferably, the desired mashing temperature is attained within 15 minutes, or more preferably within 10 minutes, such as within 9, 8, 7, 6, 5, 4, 3, 2 minutes or even more preferably within 1 minute after the mash forming, or most preferably the desired mashing temperature is attained at the mash forming. The temperature profile of the mashing process may be a profile from a conventional mashing process wherein the temperatures are set to achieve optimal degradation of the grist dry matter by the malt enzymes.

The mashing process generally applies a controlled stepwise increase in temperature, where each step favors one enzymatic action over the other, eventually degrading proteins, cell walls and starch. Mashing temperature profiles are generally known in the art. In the present invention the saccharification (starch degradation) step in the mashing process is preferably performed between 60° C. and 66° C., more preferably between 61° C. and 65° C., even more preferably between 62° C. and 64° C., and most preferably between 63° C. and 64° C. In a particular embodiment of the present invention the saccharification temperature is 64° C.

In a preferred embodiment of the first aspect, the mashing comprises an incubation step at a temperature of 65° C. or higher for at least 20 minutes; preferably at a temperature of 67° C. or higher for at least 20 minutes; even more preferably at 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C. or most preferably at a temperature of 87° C. or higher for at least 20 minutes.

In one aspect, the pH of the mash is in the range of about 4.6 to about 6.4. In another aspect, the pH is in the range of about 4.6 to 6.2, such as in the range between pH about 4.8 to about 6.0, preferably in the range between pH about 5.0 to about 6.0, more preferably in the range between pH about 5.0 to about 5.6, even more preferably in the range between pH about 5.0 to about 5.4.

In another preferred embodiment of the first aspect, the pH of the mash is about 4.6 to about 6.4.

The malt is preferably derived from one or more of the grains selected from the list consisting of corn, barley, wheat, rye, sorghum, millet and rice. Preferably, the malt is barley malt. The grist preferably comprises from 0.5% to 99%, preferably from 1% to 95%, more preferably from 5% to 90%, even more preferably from 10% to 80% malt.

Preferably the wort of the first aspect has more than 80% glucose, compared to the total carbohydrate content of the wort.

In addition to malted grain, the grist may preferably comprise adjunct such as unmalted corn, or other unmalted grain, such as barley, wheat, rye, oat, corn, rice, milo, millet and/or sorghum, or raw and/or refined starch and/or sugar containing material derived from plants like wheat, rye, oat, corn, rice, milo, millet, sorghum, potato, sweet potato, cassava, tapioca, sago, banana, sugar beet and/or sugar cane. For the invention, adjuncts may be obtained from tubers, roots, stems, leaves, legumes, cereals and/or whole grain. Preferred is adjunct obtained from corn and/or rice, more preferred the adjunct is rice starch, corn starch and/or corn grits. The mash preferably comprises from 1% to 60%, preferably from 5% to 45%, more preferably from 10% to 40% adjunct starch. The adjunct may also comprise readily fermentable carbohydrates such as sugars or syrups and may be added to the malt mash before, during or after the mashing process of the invention but is preferably added after the mashing process. Prior to forming the mash, the malt and/or adjunct is preferably milled and most preferably dry or wet milled. In one aspect, the adjunct has a high gelatinization temperature, more particularly higher onset gelatinization temperature for e.g. corn, rice and sorghum. In one aspect, the adjunct is gelatinized prior to mashing. In another aspect, the adjunct is not gelatinized prior to mashing.

In one embodiment, the mash is comprised of at least 20% of adjuncts which have a starch gelatinization temperature, preferably onset gelatinization temperature, of at least 65° C. In another aspect, the mash is comprised of at least 25%, e.g. at least 30%, at least 35%, at least 40%, at least 45%, at least 50%, at least 55%, at least 60% such as at least 65% adjuncts which have a starch gelatinization temperature, preferably onset gelatinization temperature, of at least 65° C.

In one embodiment, the mash comprises at least 10% unmalted grains compared to the total grist. In another aspect, the mash comprises at least 15%, e.g. at least 20%, at least 25%, at least 30%, at least 35%, at least 40%, at least 45%, at least 50%, at least 55%, such as at least 60% unmalted grains.

Preferably, the adjunct comprises corn. In another aspect of the invention, the mash comprises at least 20% of corn adjunct. In one aspect, the mash comprises at least 25%, e.g., at least 30%, at least 35%, at least 40%, at least 45%, at least 50%, at least 55%, such as at least 60% of corn adjunct; preferably, the corn adjunct is ungelatinized when added to the mash.

In another preferred embodiment, the adjunct comprises rice. In another aspect of the invention, the mash comprises at least 20% of rice adjunct. In one aspect, the mash comprises at least 25%, e.g., at least 30%, at least 35%, at least 40%, at least 45%, at least 50%, at least 55%, such as at least 60% of rice adjunct; preferably, the rice adjunct is ungelatinized when added to the mash.

In a preferred embodiment, practicing the method of the invention leads to additional glucose formation at temperatures between 65° C. to 90° C. In one aspect, the additional glucose formed at temperatures between 65° C. to 90° C. is at least 1% e.g., at least 2%, at least 3%, at least 4%, at least 5%, at least 6%, at least 7%, at least 8%, at least 9%, at least 10% when compared to the wort produced in the absence of the such a glucoamylase.

Preferably, practicing the method of the invention leads to decreased concentration of maltose in the wort. In one aspect, the concentration of maltose is decreased by at least 0.5%, e.g., at least 1% e.g., at least 2%, at least 3%, at least 4%, at least 5%, when compared to the wort produced in the absence of such a glucoamylase.

In a preferred embodiment, the thermostable variant glucoamylase is exogenously supplied and/or present in the mash. In one aspect, the glucoamylase is introduced at the beginning of mashing. In another aspect, the glucoamylase is introduced during mashing. In another aspect, the glucoamylase is introduced under lautering.

It is evident from the examples below, that the saccharification or mashing step(s) in brewing can be shortened, when the thermostable glucoamylase variant of the first aspect of the invention is employed, which is clearly of commercial interest as it will lower both capacity costs and energy costs. The invention allows the brewer to reach a desired pre-defined target glucose concentration faster in other words.

Accordingly, a preferred embodiment of the invention relates to the method of the first aspect, wherein a target glucose concentration is reached in a shorter saccharification time compared to when a less thermostable AMG enzyme than that of claim 1 has been added.

Preferably, the methods of the invention lead to shortened mashing times; more preferably, the methods lead to decrease in mashing time by at least 5 minutes e.g., at least 10 minutes, at least 15 minutes, at least 20 minutes, at least 25 minutes more preferably by at least 30 minutes compared to the methods done in the absence of adding a thermostable glucoamylase variant of the invention.

### Glucoamylases

Glucoamylases are also called amyloglucosidases, and Glucan 1,4-alpha-glucosidase (EC 3.2.1.3), more commonly they are referred to as AMGs.

According to the present invention, different types of amyloglucosidases may be used as parent for the generation of a thermostable amyloglucosidase variant, e.g, the amyloglucosidase may be a polypeptide that is encoded by a DNA sequence that is found in a fungal strain of Aspergillus, Rhizopusor, Talaromyces (Rasamsonia) or Penicillium; preferably the DNA sequence that is found in a fungal strain of Penicillium, even more preferably the DNA sequence that is found in a fungal strain of Penicillium oxysporum, Penicillium oxalicum, Penicillium miczynskii, Penicillium russellii or Penicillium glabrum. Preferably, the parent glucoamylase is from a species of Penicillium, preferably from Penicillium oxicalum, Penicillium miczynskii, Penicillium russellii or Penicillium glabrum.

Examples of other suitable fungi include Aspergillus niger, Aspergillus awamori, Aspergillus oryzae, Rhizopus delemar, Rhizopus niveus, Rhizopus oryzae and Talaromyces emersonii (Rasamsonia emersonii).

Below is shown the %-identity between the AMG amino acid sequences
aligned in FIG. 1, and also provided in the sequence list:

| | | | | | | | | | |
|---|---|---|---|---|---|---|---|---|---|
| P_oxalicum | 100.00 | 99.83 | 98.99 | 98.82 | 96.64 | 95.97 | 77.07 | 77.12 | 74.32 |
| AMG_NL | 99.83 | 100.00 | 99.16 | 98.99 | 96.81 | 96.13 | 77.07 | 77.12 | 74.32 |
| AMG_anPAV498 | 98.99 | 99.16 | 100.00 | 99.83 | 97.65 | 96.97 | 76.73 | 76.95 | 73.82 |
| AMG_JPO001 | 98.82 | 98.99 | 99.83 | 100.00 | 97.82 | 97.14 | 76.73 | 76.95 | 73.82 |
| AMG_JPO124 | 96.64 | 96.81 | 97.65 | 97.82 | 100.00 | 99.33 | 77.07 | 77.12 | 74.32 |
| AMG_JPO-172 | 95.97 | 96.13 | 96.97 | 97.14 | 99.33 | 100.00 | 76.73 | 76.78 | 73.99 |
| P_miczynskii | 77.07 | 77.07 | 76.73 | 76.73 | 77.07 | 76.73 | 100.00 | 94.75 | 80.51 |
| P_russellii | 77.12 | 77.12 | 76.95 | 76.95 | 77.12 | 76.78 | 94.75 | 100.00 | 79.66 |
| P_glabrum | 74.32 | 74.32 | 73.82 | 73.82 | 74.32 | 73.99 | 80.51 | 79.66 | 100.00 |

Thermostable variants of the PoAMG have been generated (see table 2 below). In a preferred embodiment, the mature thermostable glucoamylase variant of the invention comprises one or more or all of the combinations of amino acid substitutions listed in table 2 below.

In a preferred embodiment, the mature variant of the invention comprises at least one amino acid modification in one or more or all of the positions corresponding to positions 1, 2, 4, 6, 7, 11, 31, 34, 50, 65, 79, 103, 132, 327, 445, 447, 481, 484, 501, 539, 566, 568, 594 and 595 in SEQ ID NO:1; preferably the at least one amino acid modification comprises a substitution in one or more or all of the positions corresponding to positions 1, 2, 4, 11, 65, 79 and 327 in SEQ ID NO: 1, preferably the at least one amino acid modification comprises a substitution in one or more or all of the positions corresponding to R1A, P2N, P4S, P11F, T65A, K79V and Q327F in SEQ ID NO:1; or preferably the at least one amino acid modification comprises a substitution in one or more or all of the positions corresponding to positions 1, 6, 7, 31, 34, 79, 103, 132, 445, 447, 481, 566, 568, 594 and 595 in SEQ ID NO:1, preferably the at least one amino acid modification comprises a substitution in one or more or all of the positions corresponding to R1A, G6S, G7T, R31F, K34Y, K79V, S103N, A132P, D445N, V447S, S481P, D566T, T568V, Q594R and F595S in SEQ ID NO: 1; or preferably the at least one amino acid modification comprises a substitution in one or more or all of the positions corresponding to positions 1, 6, 7, 31, 34, 50, 79, 103, 132, 445, 447, 481, 484, 501, 539, 566, 568, 594 and 595 in SEQ ID NO:1, preferably the at least one amino acid modification comprises a substitution in one or more or all of the positions corresponding to R1A, G6S, G7T, R31F, K34Y, E50R, K79V, S103N, A132P, D445N, V447S, S481P, T484P, E501A, N539P, D566T, T568V, Q594R and F595S in SEQ ID NO:1; or preferably the at least one amino acid modification comprises a substitution in one or more or all of the positions corresponding to positions 1, 6, 7, 31, 34, 50, 79, 103, 132, 445, 447, 481, 484, 501, 539, 566, 568, 594 and 595 in SEQ ID NO:1, preferably the at least one amino acid modification comprises a substitution in one or more or all of the positions corresponding to R1A, G6S, G7T, R31F, K34Y, E50R, K79V, S103N, A132P, D445N, V447S, S481P, T484P, E501A, N539P, D566T, T568V, Q594R and F595S in SEQ ID NO:1; or preferably the at least one amino acid modification comprises a substitution in one or more or all of the positions corresponding to positions 1, 6, 7, 31, 34, 50, 79, 103, 132, 445, 447, 481, 484, 501, 539, 566, 568, 594 and 595 in SEQ ID NO:1, preferably the at least one amino acid modification comprises a substitution in one or more or all of the positions corresponding to R1A, G6S, G7T, R31F, K34Y, E50R, K79V, S103N, A132P, D445N, V447S, S481P, T484P, E501A, N539P, D566T, T568V, Q594R and F595S in SEQ ID NO:1.

The thermostability improvements (Td) of the variants in table 2 are listed in Table 3, where the Td of the PoAMG variant denoted “anPAV498” (the parent) was set to zero. In a preferred embodiment, the mature thermostable variant of the invention has a thermostability improvement (Td) over its parent of at least 5° C., preferably at least 6° C., 7° C. or 8° C., preferably determined as exemplified herein.

In another preferred embodiment, the mature thermostable variant of the invention has a relative activity at 91° C. of at least 150, preferably at least 200, more preferably at least 250, most preferably at least 300 compared to its parent.

### Additional Enzymes

In a preferred embodiment of the first aspect, one or more additional enzyme is added to the mash, said additional enzyme may be selected from the group consisting of a alpha amylase, maltogenic amylase, raw-starch degrading alpha amylase, beta amylase, aminopeptidase, carboxypeptidase, catalase, cellobiose oxidase, cellulase, chitinase, cutinase, cyclodextrin glycosyltransferase, deoxyribonuclease, esterase, glucan 1,4-alpha-maltotetrahydrolase, glucanase, beta glucanase, galactanase, alpha-galactosidase, beta-galactosidase, glucose oxidase, alpha-glucosidase, beta-glucosidase, haloperoxidase, hemicellulytic enzyme, invertase, laccase, lipase, mannanase, mannosidase, oxidase, pectinolytic enzymes, peptidoglutaminase, peroxidase, phospholipase, phytase, polyphenoloxidase, protease, pullulanase, raw starch degrading alpha amylase, ribonuclease, transglutaminase, and xylanase.

### Raw Starch Degrading Alpha-Amylase

As used herein, a “raw starch degrading alpha-amylase” refers to an enzyme that can directly degrade raw starch granules below the gelatinization temperature of starch.

Examples of raw starch degrading alpha-amylases include the ones disclosed in WO 2005/003311, U.S. Patent Publication no. 2005/0054071, and U.S. Pat. No. 7,326,548. Examples also include those enzymes disclosed in Table 1 to 5 of the examples in U.S. Pat. No. 7,326,548, in U.S. Patent Publication no. 2005/0054071 (Table 3 on page 15), as well as the enzymes disclosed in WO 2004/020499 and WO 2006/06929 and WO 2006/066579.

In one embodiment, the raw starch degrading alpha-amylase is a GH13_1 amylase.

In one embodiment, the raw starch degrading alpha-amylase enzyme has at least 70%, e.g. at least 71%, e.g. at least 72%, e.g. at least 73%, e.g. at least 74%, e.g. at least 75%, e.g. at least 76%, e.g. at least 77%, e.g. at least 78%, e.g. at least 79%, e.g., at least 80%, e.g. at least 81%, e.g. at least 82%, e.g. at least 83%, e.g. at least 84%, e.g., at least 85%, e.g. at least 86%, e.g. at least 87%, e.g. at least 88%, e.g. at least 89%, e.g., at least 90%, e.g., at least 91%, e.g., at least 92%, e.g., at least 93%, e.g., at least 94%, e.g., at least 95%, e.g. at least 96%, e.g., at least 97%, e.g., at least 98%, e.g., at least 99% identity to the raw starch degrading alpha-amylase shown in EP U.S. Pat. No. 2,981,170 (Novozymes A/S) or in SEQ ID NO: 11 herein.

### Amylases

Alpha-Amylases (alpha-1,4-glucan-4-glucanohydrolases, EC. 3.2.1.1) constitute a group of enzymes which catalyze hydrolysis of starch and other linear and branched 1,4-glucosidic oligo- and polysaccharides.

A number of alpha-amylases are referred to as Termamyl™, Termamyl® SC and “Termamyl™-like alpha-amylases” and are known from, e.g., WO 90/11352, WO 95/10603, WO 95/26397, WO 96/23873 and WO 96/23874.

Another group of alpha-amylases are referred to as Fungamyl™ and “Fungamyl™-like alpha-amylases”, which are alpha-amylases related to the alpha-amylase derived from Aspergillus oryzae disclosed in WO 01/34784.

### Proteases

Suitable proteases include microbial proteases, such as fungal and bacterial proteases. Preferred proteases are acidic proteases, i.e., proteases characterized by the ability to hydrolyze proteins under acidic conditions below pH 7. The proteases are responsible for reducing the overall length of high-molecular-weight proteins to low-molecular-weight proteins in the mash. The low-molecular-weight proteins are a necessity for yeast nutrition and the high-molecular-weight-proteins ensure foam stability. Thus it is well-known to the skilled person that protease should be added in a balanced amount which at the same time allows ample free amino acids for the yeast and leaves enough high-molecular-weight-proteins to stabilize the foam. In one aspect, the protease activity is provided by a proteolytic enzymes system having a suitable FAN generation activity including endo-proteases, exopeptidases or any combination hereof, preferably a metallo-protease. Preferably, the protease has at least 50%, more preferably at least 60%, more preferably at least 70%, more preferably at least 80%, more preferably at least 85%, more preferably at least 90%, more preferably at least 91%, more preferably at least 92%, more preferably at least 93%, more preferably at least 94%, more preferably at least 95% more preferably at least 96%, more preferably at least 97% more preferably at least 98%, and most preferably at least 99% or even 100% identity to the amino acid sequence shown in SEQ ID NO:6 described in WO9967370. In another aspect, the protease is Neutrase® available from Novozymes A/S. Proteases may be added in the amounts of, 0.0001-1000 AU/kg DS, preferably 1-100 AU/kg DS and most preferably 5-25 AU/kg dry weight cereal(s). The proteolytic activity may be determined by using denatured hemoglobin as substrate. In the Anson-Hemoglobin method for the determination of proteolytic activity, denatured hemoglobin is digested, and the undigested hemoglobin is precipitated with trichloroacetic acid (TCA). The amount of the TCA soluble product is determined by using phenol reagent, which gives a blue color with tyrosine and tryptophan. One Anson Unit (AU) is defined as the amount of enzyme which under standard conditions (i.e. 25° C., pH 7.5 and 10 min. reaction time) digests hemoglobin at an initial rate such that there is liberated an amount of TCA soluble product per minute which gives the same colour with phenol reagent as one milliequivalent of tyrosine.

### Enzyme Compositions

The mature thermostable variant glucoamylase of the invention as well as any additional enzyme(s) may be added in any suitable form, such as, e.g., in the form of a liquid, in particular a stabilized liquid, or it may be added as a substantially dry powder or granulate.

Granulates may be produced, e.g., as disclosed in U.S. Pat. Nos. 4,106,991 and 4,661,452. Liquid enzyme preparations may, for instance, be stabilized by adding a sugar or sugar alcohol or lactic acid according to established procedures. Other enzyme stabilizers are well-known in the art.

The enzyme(s) may be added in any suitable manner, such as individual components (separate or sequential addition of the enzymes) or addition of the enzymes together in one step or one composition.

Granulates and agglomerated powders may be prepared by conventional methods, e.g., by spraying the enzymes onto a carrier in a fluid-bed granulator. The carrier may consist of particulate cores having a suitable particle size. The carrier may be soluble or insoluble, e.g. a salt (such as NaCl or sodium sulfate), a sugar (such as sucrose or lactose), a sugar alcohol (such as sorbitol), starch, rice, corn grits, or soy.

One aspect of the invention relates to mashing or brewing compositions comprising a mature thermostable variant of a parent glucoamylase as defined in the first aspect of the invention and preferred embodiments thereof. Preferably, the mashing or brewing compositions also comprise one or more additional enzyme selected from the group consisting of a alpha amylase, maltogenic amylase, raw-starch degrading alpha amylase, beta amylase, aminopeptidase, carboxypeptidase, catalase, cellobiose oxidase, cellulase, chitinase, cutinase, cyclodextrin glycosyltransferase, esterase, deoxyribonuclease, glucan 1,4-alpha-maltotetrahydrolase, glucanase, beta glucanase, galactanase, alpha-galactosidase, beta-galactosidase, glucose oxidase, alpha-glucosidase, beta-glucosidase, haloperoxidase, hemicellulytic enzyme, invertase, laccase, lipase, mannanase, mannosidase, oxidase, pectinolytic enzymes, peptidoglutaminase, peroxidase, phospholipase, phytase, polyphenoloxidase, protease, pullulanase, ribonuclease, transglutaminase, and xylanase.

### EXAMPLES

### Example 1: Construction of PoAMG Libraries

PoAMG libraries were constructed as follows:

A forward or reverse primer having NNK or desired mutation(s) at target site(s) with 15 bp overlaps each other were designed. Inverse PCR, which means amplification of entire plasmid DNA sequences by inversely directed primers, were carried out with appropriate template plasmid DNA (e.g. plasmid DNA containing JPO-0001 gene) by the following conditions. The resultant PCR fragments were purified by QIAquick Gel extraction kit [QIAGEN], and then introduced into Escherichia coli ECOS Competent E. coli DH5a [NIPPON GENE CO., LTD.]. The plasmid DNAs were extracted from E. coli transformants by MagExtractor plasmid extraction kit [TOYOBO], and then introduced into A. niger competent cells.

PCR reaction mix:
PrimeSTAR Max DNA polymerase [TaKaRa]

| | |
|---|---|
| Total 25 μl | |
| 1.0 μl | Template DNA (1 ng/μl) |
| 9.5 μl | H₂O |
| 12.5 μl | 2x PrimeSTAR Max pre-mix |
| 1.0 μl | Forward primer (5 μM) |
| 1.0 μl | Reverse primer (5 μM) |

PCR program:

98° C./2 min
25x (98° C./10 sec, 60° C./15 sec, 72° C./2 min)
10° C./hold

### Example 2: Screening for Better Thermostability

B. subtilis libraries constructed as in Example 1 were fermented in either 96-well or 24-well MTP containing COVE liquid medium (2.0 g/L sucrose, 2.0 g/L iso-maltose, 2.0 g/L maltose, 4.9 mg/L, 0.2 ml/L 5N NaOH, 10 ml/L COVE salt, 10 ml/L 1M acetamide), 32° C. for 3 days. Then, AMG activities in culture supernatants were measured at several temperatures by pNPG assay described as follows.

pNPG Thermostability Assay:

The culture supernatants containing desired enzymes was mixed with same volume of pH 5.0 200 mM NaOAc buffer. Twenty microliter of this mixture was dispensed into either 96-well plate or 8-strip PCR tube, and then heated by thermal cycler at various temperatures for 30 min. Those samples were mixed with 10 μl of substrate solution containing 0.1% (w/v) pNPG [wako] in pH 5.0 200 mM NaOAc buffer and incubated at 70° C. for 20 min for enzymatic reaction. After the reaction, 60 μl of 0.1M Borax buffer was added to stop the reaction. Eighty microliter of reaction supernatant was taken out and its OD₄₀₅ value was read by photometer to evaluate the enzyme activity.

TABLE 1a
Lists of the relative activity of PoAMG variants
when compared with their parent anPAV498 or
JPO-0001 (anPAV498 w. leader-/propeptide)

| Name | Relative activity of 80° C./75° C. (%) |
|---|---|
| anPAV498 | 17% |
| JPO-004 | 32% |
| JPO-005 | 15% |
| JPO-006 | 16% |
| JPO-007 | 3% |
| AnPav498 | 13% |
| JPO-009 | 16% |
| JPO-011 | 15% |
| JPO-012 | 15% |
| JPO-013 | 17% |
| JPO-020 | 20% |
| Name | Relative activity of 80° C./70° C. (%) |
| JPO-001 | 10% |
| JPO-004 | 29% |
| JPO-009 | 13% |
| JPO-014 | 21% |
| JPO-020 | 16% |
| JPO-021 | 30% |
| JPO-052 | 33% |
| Name | Relative activity of 79° C./70° C. (%) |
| JPO-001 | 23% |
| JPO-021 | 46% |
| JPO-022 | 39% |
| JPO-023 | 44% |
| JPO-025 | 51% |
| JPO-027 | 49% |
| JPO-029 | 37% |
| Name | Relative activity of 77° C./70° C. (%) |
| JPO-001 | 72% |
| JPO-029 | 82% |
| JPO-047 | 80% |
| JPO-048 | 90% |
| JPO-049 | 84% |
| JPO-050 | 86% |
| JPO-064 | 87% |
| name | Relative activity of 79° C./77° C. (%) |
| JPO-001 | 36% |
| JPO-029 | 51% |
| JPO-047 | 45% |
| JPO-048 | 81% |
| JPO-049 | 53% |
| JPO-050 | 58% |
| JPO-064 | 65% |
| Name | Relative activity of 79° C./77° C. (%) |
| JPO-001 | 41% |
| JPO-021 | 60% |
| JPO-022 | 48% |
| JPO-023 | 57% |
| JPO-025 | 56% |
| JPO-027 | 64% |
| JPO-029 | 66% |
| JPO-047 | 50% |
| JPO-048 | 72% |
| JPO-051 | 82% |
| JPO-058 | 73% |
| JPO-062 | 72% |
| JPO-063 | 85% |
| JPO-064 | 83% |

TABLE 1b
Lists of the relative activity of PoAMG variants
when compared with their parent JPO-022

| Name | Relative activity of 77° C./70° C. (%) |
|---|---|
| JPO-022 | 60% |
| JPO-027 | 67% |
| JPO-042 | 8% |
| JPO-044 | 86% |
| JPO-045 | 67% |
| JPO-046 | 48% |
| JPO-022 | 76% |
| JPO-023 | 75% |
| JPO-025 | 80% |
| JPO-027 | 84% |
| JPO-058 | 92% |
| JPO-059 | 88% |
| JPO-060 | 86% |
| JPO-061 | 83% |
| JPO-062 | 87% |
| Name | Relative activity of 79° C./77° C. (%) |
| JPO-022 | 49% |
| JPO-023 | 51% |
| JPO-025 | 52% |
| JPO-027 | 58% |
| JPO-058 | 69% |
| JPO-059 | 36% |
| JPO-060 | 41% |
| JPO-061 | 44% |
| JPO-062 | 57% |

TABLE 1c
Lists of the relative activity of PoAMG variants when compared
with their parent JPO-063 at different temperatures

| Name | Relative activity of 79° C./77° C. (%) |
|---|---|
| JPO-063 | 91% |
| JPO-066 | 96% |
| JPO-071 | 89% |
| JPO-072 | 84% |
| JPO-074 | 103% |
| JPO-075 | 86% |
| JPO-076 | 92% |
| JPO-077 | 95% |
| JPO-078 | 88% |
| JPO-079 | 100% |
| Name | Relative activity of 84° C./80° C. (%) |
| JPO-063 | 16% |
| JPO-065 | 26% |
| JPO-067 | 21% |
| JPO-070 | 12% |
| JPO-071 | 13% |
| JPO-074 | 32% |
| JPO-081 | 17% |
| JPO-082 | 24% |
| JPO-083 | 46% |
| JPO-084 | 26% |
| JPO-044 | 37% |
| Name | Relative activity of 82° C./70° C. (%) |
| JPO-063 | 21% |
| JPO-093 | 43% |
| JPO-081 | 25% |
| JPO-088 | 39% |
| JPO-094 | 38% |
| JPO-096 | 38% |
| JPO-106 | 53% |
| Name | Relative activity of 83° C./80° C. (%) |
| JPO-063 | 46% |
| JPO-051 | 44% |
| JPO-096 | 64% |
| JPO-106 | 88% |
| JPO-110 | 81% |
| JPO-111 | 100% |
| JPO-112 | 86% |
| JPO-113 | 83% |
| JPO-114 | 47% |
| JPO-115 | 90% |

TABLE 1d
List of the relative activity of PoAMG variants
when compared with their parent JPO-096

| Name | Relative activity of 83° C./70° C. (%) |
|---|---|
| JPO-082 | 53% |
| JPO-088 | 70% |
| JPO-091 | 69% |
| JPO-092 | 65% |
| JPO-093 | 62% |
| JPO-094 | 74% |
| JPO-095 | 69% |
| JPO-096 | 67% |
| JPO-097 | 65% |
| JPO-098 | 65% |
| Name | Relative activity of 83° C./80° C. (%) |
| JPO-051 | 20% |
| JPO-096 | 43% |
| JPO-109 | 51% |
| JPO-126 | 33% |
| JPO-129 | 48% |
| JPO-130 | 18% |
| JPO-131 | 51% |
| JPO-132 | 34% |

TABLE 1e
List of the relative activity of PoAMG variants
when compared with their parents JPO-129

| Name | Relative activity of 84° C./80° C. (%) |
|---|---|
| JPO-129 | 62% |
| JPO-156 | 51% |
| JPO-160 | 34% |
| JPO-161 | 41% |
| JPO-162 | 49% |
| JPO-163 | 21% |
| JPO-164 | 57% |
| JPO-165 | 77% |

TABLE 1f
List of the relative activity of PoAMG variants
when compared with their parent JPO-166

| Name | Relative activity of 84° C./75° C. (%) |
|---|---|
| JPO-166 | 19% |
| JPO-167 | 66% |
| JPO-168 | 58% |
| JPO-169 | 53% |
| JPO-171 | 47% |
| JPO-172 | 98% |

TABLE 2
Amino acid substitutions in the variants of the PoAMG mature sequence

| Name | Amino acid substitutions |
|---|---|
| PoAMG | The wildtype mature AMG from Penicillium (SEQ ID NO: 1) |
| AMG NL | K79V |
| anPAV498 | P2N P4S P11F T65A K79V Q327F |
| JPO-001 | R1A P2N P4S P11F T65A K79V Q327F |
| JPO-018 | D75N R77D A78Q |
| JPO-019 | D75S R77G A78W V79D F80Y |
| JPO-023 | R1A K34Y S103N |
| JPO-024 | R1A K34Y D445N V447S |
| JPO-025 | R1A K34Y Y504T |
| JPO-026 | R1A S103N D445N V447S |
| JPO-027 | R1A S103N Y504T |
| JPO-028 | R1A D445N V447S Y504T |
| JPO-029 | R1A K34Y S103N D445N V447S |
| JPO-044 | R1A K34Y S103N D445N V447S E501V Y504T |
| JPO-047 | R1A K34Y S103N Y504T |
| JPO-048 | R1A K34Y S103N D445N V447S D566T |
| JPO-049 | R1A K34Y S103N Q594R F595S |
| JPO-050 | R1A K34Y S103N Y504T Q594R F595S |
| JPO-051 | R1A K34Y S103N D445N V447S Y504T Q594R F595S |
| JPO-052 | R1A S105L |
| JPO-053 | R1A S105E |
| JPO-055 | R1A A132R |
| JPO-058 | R1A K34Y S105L Y504T Q594R F595S |
| JPO-059 | R1A K34Y S103N S105L Y504T Q594R F595S |
| JPO-060 | R1A K34Y S103N S105L Y504T Q594R F595S |
| JPO-061 | R1A K34Y S103N S105L Y504T D566T Q594R F595S |
| JPO-062 | R1A K34Y S103N S105L D445N V447S Y504T D566T Q594R F595S |
| JPO-063 | R1A K34Y S103N S105L A132R D445N V447S Y504T D566T Q594R F595S |
| JPO-064 | R1A K34Y S103N S105L D445N V447S D566T Q594R F595S |
| JPO-065 | R1A K34Y S103N S105L A132R D445N V447S E501V Y504T D566T Q594R R1A |
| | F595S |
| JPO-066 | R1A K34Y S103N A132R D445N V447S Y504T D566T Q594R F595S |
| JPO-069 | R1A K34Y S103N S105L A132R D445N V447S Y504T D566T V592T |
| JPO-071 | R1A G6S G7T K34Y S103N S105L A132R D445N V447S Y504T D566T Q594R |
| | R1A F595S |
| JPO-074 | R1A K34Y S103N P107L A132R D445N V447S Y504T D566T Q594R F595S |
| JPO-083 | R1A G6S G7T K34Y S103N P107L A132R D445N V447S Y504T D566T Q594R |
| | R1A F595S |
| JPO-084 | R1A G6S G7T K34Y S103N P107L A132R D445N V447S Y504T D566T V592T |
| | R1A Q594R F595S |
| JPO-091 | R1A G6S R7T K34Y S103N P107L A132P D445N V447S Y504T D566T Q594R |
| | F595S |
| JPO-092 | R1A G6S G7T K34Y S103N P107L A132R D445N V447S Y504T D566T T568V |
| | Q594R F595S |
| JPO-093 | R1A G6S G7T K34Y S103N P107L A132P D445N V447S Y504T D566T T568V |
| | Q594R F595S |
| JPO-094 | R1A G6S G7T K34Y S103N P107L A132R D445N V447S S481P Y504T D566T |
| | Q594R F595S |
| JPO-095 | R1A G6S G7T K34Y S103N P107L A132R D445N V447S S481P Y504T D566T |
| | T568V Q594R F595S |
| JPO-096 | R1A G6S G7T K34Y S103N P107L A132P D445N V447S D566T T568V Q594R |
| | F595S |
| JPO-097 | R1A G6S G7T K34Y S103N P107L T110W A132P D445N V447S Y504T D566T |
| | T568V Q594R F595S |
| JPO-098 | R1A G6S G7T K34Y E50R S103N P107L A132P D445N V447S Y504T D566T |
| | T568V Q594R F595S |
| JPO-105 | R1A G6S G7T K34Y S103N P107L A132P D445N V447S E501V Y504T |
| JPO-106 | R1A G6S G7T R31F K34Y S103N P107L A132P D445N V447S Y504T D566T |
| | T568V Q594R F595S |
| JPO-108 | R1A G6S G7T R31F K34Y S103N P107L A132P D445N V447S S481P Y504T |
| | D566T T568V Q594R F595S |
| JPO-109 | R1A G6S G7T K34Y E50R S103N P107L A132P D445N V447S S481P Y504T |
| | D566T T568V Q594R F595S |
| JPO-111 | R1A G6S G7T R31F K34Y S103N P107L A132P D445N V447S S481P E501V |
| | Y504T D566T T568V Q594R F595S |
| JPO-112 | R1A G6S G7T R31F K34Y S103N P107L A132P D445N V447S S481P D566T |
| | T568V Q594R F595S |
| JPO-114 | R1A K34Y D75N R77D A78Q S103N R138L D445N V447S Y504T Q594R F595S |
| JPO-115 | R1A G6S G7T R31F K34Y D75N R77D A78Q S103N P107L A132P D445N |
| | V447S S481P Y504T D566T T568V Q594R F595S |
| JPO-124 | R1A G6S G7T R31F K34Y S103N A132P D445N V447S S481P D566T T568V |
| | Q594R F595S |
| JPO-125 | R1A G6S G7T K34Y E50R S103N A132P D445N V447S S481P D566T T568V |
| | Q594R F595S |
| JPO-126 | R1A R31F K34Y D75N R77D A78Q S103N R138L D445N V447S Y504T Q594R |
| | F595S |
| JPO-127 | R1A K34Y D75N R77D A78Q S103N R138L D445N V447S Q594R F595S |
| JPO-128 | R1A G6S G7T R31F K34Y S103N A132P D445N V447S |
| JPO-129 | R1A G6S G7T R31F K34Y E50R S103N A132P D445N V447S S481P D566T |
| | T568V Q594R F595S |
| JPO-130 | R1A K34Y E50R D75N R77D A78Q S103N R138L D445N V447S Q594R F595S |
| JPO-131 | R1A G6S G7T R31F K34Y E50R D75N R77D A78Q S103N A132P D445N V447S |
| | S481P D566T Q594R F595S |
| JPO-132 | R1A R31F K34Y E50R D75N R77D A78Q S103N R138L D445N V447S Q594R |
| | F595S |
| JPO-133 | R1A G6S G7T R31F K34Y E50R D75N R77D A78Q S103N A132P R138L D445N |
| | V447S S481P D566T Q594R F595S |
| JPO-138 | R1A R135S |
| JPO-143 | R1A G6S G7T R31F K34Y E50R S103N A132P D445N V447S S481P E501L |
| | D566T T568V Q594R F595S |
| JPO-154 | R1A G6S G7T R31F K34Y S103N A132P R138G D445N V447S S481P D566T |
| | T568V Q594R F595S |
| JPO-155 | R1A G6S G7T R31F K34Y S103N A132P R138L D445N V447S S481P D566T |
| | T568V Q594R F595S |
| JPO-156 | R1A G6S G7T R31F K34Y S103N A132P R138P D445N V447S S481P D566T |
| | T568V Q594R F595S |
| JPO-167 | R1A G6S G7T R31F K34Y E50R S103N A132P S379P D445N V447S S481P |
| | E501A D566T T568V Q594R F595S |
| JPO-168 | R1A G6S G7T R31F K34Y E50R S103N A132P D445N V447S S481P T484P |
| | E501A D566T T568V Q594R F595S |
| JPO-169 | R1A G6S G7T R31F K34Y E50R S103N A132P D445N V447S S481P E501A |
| | N539P D566T T568V Q594R F595S |
| JPO-171 | R1A G6S G7T R31F K34Y E50R S103N A132P S379P D445N V447S S481P |
| | T484P E501A D566T T568V Q594R F595S |
| JPO-172 | R1A G6S G7T R31F K34Y E50R S103N A132P D445N V447S S481P T484P |
| | E501A N539P D566T T568V Q594R F595S |

### Example 3: Fermentation of the Aspergillus niger

Aspergillus niger strains were fermented on a rotary shaking table in 500 ml baffled flasks containing 100 ml MU1 with 4 ml 50% urea at 220 rpm, 30° C. The culture broth was centrifuged (10,000×g, 20 min) and the supernatant was carefully decanted from the precipitates.

### Example 4: Purification of PoAMG (JPO-001) Variants

PoAMG variants were purified by cation exchange chromatography. The peak fractions of each were pooled individually and dialyzed against 20 mM sodium acetate buffer pH 5.0, and then the samples were concentrated using a centrifugal filter unit (Vivaspin Turbo 15, Sartorius). Enzyme concentrations were determined by A280 value.

### Example 5: Thermostability Determination (TSA)

Purified enzyme was diluted with 50 mM sodium acetate buffer pH 5.0 to 0.5 mg/ml and mixed with equal volume of SYPRO Orange (Invitrogen) diluted with Milli-Q water. Eighteen ul of mixture solution were transfer to LightCycler 480 Multiwell Plate 384 (Roche Diagnostics) and the plate was sealed.

### Equipment Parameters of TSA:

Apparatus: LightCycler 480 Real-Time PCR System (Roche Applied Science)
Scan rate: 0.02° C./sec
Scan range: 37-96° C.
Integration time: 1.0 sec
Excitation wave length 465 nm
Emission wave length 580 nm

The obtained fluorescence signal was normalized into a range of 0 and 1. The Td was defined as the temperature at which the signal intensity was 0.5. The thermostability improvements are listed in Table 3 with Td of the PoAMG variant denoted anPAV498 as 0.

### Example 6: PoAMG Activity Assay

Maltodextrin (DE11) assay by GOD-POD method

### Substrate Solution

30 g maltodextrin (pindex #2 from MATSUTANI chemical industry Co., Ltd.)
100 ml 120 mM sodium acetate buffer, pH 5.0

### Glucose CII Test Kit (Wako Pure Chemical Industries, Ltd.)

Twenty ul of enzyme samples were mixed with 100 ul of substrate solution and incubated at set temperatures for 2 hours. The samples were cooled down on the aluminum block for 3 min then 10 ul of the reaction solution was mixed with 590 ul of 1 M Tris-HCl pH 8.0 to stop reaction. Ten ul of the solution was mixed with 200 ul of the working solution of the test kit then stand at room temperature for 15 min. The absorbance at A505 was read. The activities are listed in Table 3 as relative activity of the PoAMG variant denoted anPAV498.

TABLE 3

| | Td improvement [° C.] | Activity at 91° C. |
|---|---|---|
| Variant | (pH 5.0, anPAV498 as 0) | (anPAV498 as 100) |
| anPAV498 | — | 100 |
| JPO-001 | 1.0 | 94 |
| JPO-004 | 2.2 | — |
| JPO-009 | 0.7 | — |
| JPO-013 | 1.5 | — |
| JPO-014 | 2.3 | — |
| JPO-020 | 1.4 | 74 |
| JPO-021 | 2.5 | 113 |
| JPO-052 | 2.6 | 85 |
| JPO-053 | 0.2 | 71 |
| JPO-055 | 1.6 | 85 |
| JPO-023 | 3.6 | — |
| JPO-024 | 2.5 | — |
| JPO-025 | 3.4 | — |
| JPO-027 | 2.9 | — |
| JPO-029 | 3.7 | 191 |
| JPO-048 | 4.3 | 163 |
| JPO-051 | 5.7 | 222 |
| JPO-058 | 4.2 | 157 |
| JPO-062 | 4.2 | 159 |
| JPO-063 | 5.4 | 107 |
| JPO-064 | 4.9 | 178 |
| JPO-065 | 7.0 | 127 |
| JPO-066 | 6.5 | 178 |
| JPO-069 | 4.8 | 95 |
| JPO-071 | 6.1 | 128 |
| JPO-074 | 6.3 | 108 |
| JPO-081 | 5.5 | 213 |
| JPO-082 | 5.6 | 215 |
| JPO-089 | 6.0 | 171 |
| JPO-090 | 5.5 | 155 |
| JPO-018 | 0.6 | 84 |
| JPO-019 | 0.5 | 86 |
| JPO-044 | 6.3 | 225 |
| JPO-083 | 6.1 | 103 |
| JPO-084 | 4.4 | 66 |
| JPO-099 | 6.8 | 156 |
| JPO-091 | 6.6 | 130 |
| JPO-092 | 6.7 | 113 |
| JPO-093 | 6.8 | 132 |
| JPO-094 | 6.6 | 126 |
| JPO-095 | 6.9 | — |
| JPO-096 | 5.9 | — |
| JPO-097 | 5.2 | — |
| JPO-098 | 5.6 | — |
| JPO-112 | 8.2 | — |
| JPO-114 | 5.2 | 218 |
| JPO-115 | 8.0 | — |
| JPO-108 | 8.5 | — |
| JPO-109 | 7.2 | — |
| JPO-111 | 8.4 | — |
| JPO-124 | 8.0 | 385 |
| JPO-125 | 6.8 | 324 |
| JPO-126 | 6.6 | 268 |
| JPO-127 | 4.9 | 246 |
| JPO-129 | 8.2 | 399 |
| JPO-130 | 5.3 | 278 |
| JPO-131 | 7.9 | 367 |
| JPO-132 | 6.6 | 336 |
| JPO-138 | 6.4 | 125 |
| JPO-133 | 6.1 | 143 |
| JPO-143 | 8.8 | 280 |
| JPO-154 | 7.6 | 252 |
| JPO-155 | 8.3 | 282 |
| JPO-156 | 8.3 | 290 |
| JPO-145 | 8.2 | — |
| JPO-147 | 8.2 | — |
| JPO-150 | 8.2 | — |
| JPO-152 | 8.4 | — |
| JPO-153 | 9.0 | 399 |
| JPO-161 | 6.0 | 200 |
| JPO-165 | 8.9 | 403 |
| JPO-166 | 7.0 | 237 |
| JPO-167 | 9.1 | 387 |
| JPO-168 | 9.3 | 332 |
| JPO-169 | 9.6 | 269 |
| JPO-171 | 9.4 | 255 |
| JPO-172 | 9.9 | 432 |

### Example 7: Mashing Experiment with 100% Un-Malted Sorghum

TABLE 4
Raw material characteristics of sorghums used

| | Condensed | |
|---|---|---|
| | tannins | |
| | [mg catechin | Moisture |
| | equiv./g] | [%] |
| Sorghum low tannin | 12.23 ± 0.30 | 12.61 |
| Sorghum high tannin | 28.48 ± 1.94 | 9.57 |

Sorghum was milled using a laboratory disk mill DLFU (Bühler AG, Switzerland) with a gap distance of 0.6 mm and the obtained grist stored dry in a closed container until further use.

Mashing water was prepared by adding CaCl₂) to de-ionized water to achieve a final concentration of 100 ppm Ca²⁺-ions. The mashing water was distributed to mashing beakers in portions of 125 g and placed in a LP Electronic Mashing Device (Lochner GmbH, Germany) and subsequently pre-warmed to mash-in temperature of 55° C.

Sorghum grist was added to the mashing water at portions of 50 (low tannin sorghum) or 12.5 (low tannin) plus 37.5 (high tannin) gram per beaker, respectively. The water and grist were homogenized by stirring for 5 minutes at 150 rpm. Stirring speed was subsequently reduced to 100 ppm and after this step, enzymes were added according to Table 5 below, and the mashing process started.

TABLE 5
Enzyme dosages applied at the beginning of
mashing (each trial was done in duplicate)

| | | | | | 75% high tannin + | |
|---|---|---|---|---|---|---|
| | | | 100% low tannin | | 25% low tannin | |
| | | | sorghum | | sorghum | |
| | | EC- | Dosage [mg EP/kg grist] | | | |
| | Enzyme | number | Ref. | Test | Ref. | Test |
| SEQ ID NO: 2 | AMG NL | 3.2.1.3 | 171.4 | — | 171.4 | — |
| SEQ ID NO: 6 | AMG | 3.2.1.3 | — | 171.4 | — | 171.4 |
| | JPO-172 | | | | | |
| SEQ ID NO: 11 | Alpha | 3.2.1.1 | 20.2 | 20.2 | 20.2 | 20.2 |
| | amylase | | | | | |
| SEQ ID NO: 12 | Alpha | 3.2.1.1 | 13.9 | 13.9 | 13.9 | 13.9 |
| | amylase | | | | | |
| SEQ ID NO: 13 | Protease | 3.4.21.62 | 21.1 | 21.1 | 21.1 | 21.1 |

The following mashing regimes were applied:
Mashing regime A: 20 minutes rest at 55° C.; heating from 55 to 78° C., with a heating rate of 1° C. per minute; 60 minutes rest at 78° C.; heating from 78 to 85° C., with a heating rate of 1° C. per minute; 30 minutes rest at 85° C.; cooling to 80° C.; 15 minutes rest at 80° C.; cooling to room temperature within 10 minutes. Total time without cooling=170 minutes.
Mashing regime B: 20 minutes rest at 55° C.; heating from 55 to 85° C., with a heating rate of 1° C. per minute; 90 minutes rest at 85° C.; cooling to 80° C.; 15 minutes rest at 80° C.; cooling to room temperature within 10 minutes. Total time without cooling=170 minutes.

After mashing all mashes were adjusted for evaporation losses to the original weight of 175 g at start mashing. The mashes were then filtered at room temperature using filter-funnels equipped with MN614 Ø 240 mm (Macherey-Nagel, Germany) filter-paper. The obtained wort was immediately frozen and stored until subsequent analyses.

### Analyses:

Moisture of finely ground sorghum grist, obtained from milling with laboratory hammermill Lab mill 3100 (Perkin Elmer, Sweden) with retention sieve 0.8 mm (mm nominal diameter), was determined using a HR73 Halogen Moisture Analyzer (Mettler Toledo, Switzerland) set to drying temperature of 105° C.

The concentrations of condensed tannins in sorghums were analysed by the colorimetric vanillin HCL assay described in Dykes L. Tannin Analysis in Sorghum Grains. Methods Mol Biol. 2019; 1931:109-120. doi: 10.1007/978-1-4939-9039-9_8. PMID: 30652286.

Glucose in wort was analysed with a Dionex HPLC system ICS-5000 using a RI-101 Differential Refractive Index Detector (Thermo Scientific, USA), based on Analytica-EBC method 8.7-Fermentable carbohydrates in wort by HPLC.

Density was analysed by a DMA™ 4500M density analyser (Anton Paar, Austria) to determine the concentration of extract.

Extract yield (given as % of dry matter grist added to the mash), was calculated based on the total water in mash (incl. moisture from sorghum grist), the present extract in wort (expressed in ° Plato) and the dry matter input to the mash.

### Results:

Mashing with glucoamylase JPO-172 (SEQ ID NO: 6) resulted in significantly higher final glucose concentrations than that achieved with glucoamylase AMG NL (SEQ ID NO: 2); see Tables 5 and 6. This is very clear when comparing the glucose concentrations obtained in mashing regimes A and B with the individual glucoamylases.

Whereas the glucose concentration increased by 6.1-7.4% in mashing regime B, when AMG JPO-172 (SEQ ID NO: 6) was applied in mashing, a drop of 24-27.5% was observed for AMG NL (SEQ ID NO: 2); see Tables 8 and 9.

The overall increase in extract yield seen for both AMGs (SEQ ID NO: 2 and SEQ ID NO: 6), when mashing regime B was applied, may be ascribed to the effect of higher temperature benefiting starch solubilisation and to liquefaction by the added raw-starch degrading amylase JA126PE096 (SEQ ID NO:11) and thermostable alpha-amylase (SEQ ID NO:12).

Thus, it becomes apparent, that increasing the thermostability of the saccharifying glucoamylase to temperatures, which benefit the solubilisation and liquefaction of starches with high gelatinization temperatures, enables a simultaneous starch liquefaction (by thermostable alpha-amylases) and saccharification (by thermostable glucoamylase), making the overall process simpler and more efficient.

The combination of the thermostable alpha-amylase with thermostable glucoamylase variant JPO-172 compared to the combination of thermostable alpha amylase with thermostable glucoamylase variant AMG NL, resulted in significantly improved simultaneous liquefaction and saccharification of starch derived from un-malted sorghum in high temperature infusion mashing.

In mashing with un-malted sorghum, glucose formation by AMG JPO-172 compared to AMG NL was increased by 10.8 to 43.6%, with higher mashing temperature increasing the delta improvement. When using AMG NL, the increase in mashing temperature resulted in a decrease of glucose formation between 14.0 to 17.5%, whereas when using AMG JPO-172 an increase with increasing mashing temperature of 6.1 to 7.4% was observed.

TABLE 6
Comparison of glucose concentration in wort produced
by AMG NL or AMG JPO-172, respectively using two
sorghum grist compositions and regime A for mashing

| | Mashing regime A | | | |
|---|---|---|---|---|
| | | | 75% high tannin + | |
| | 100% low tannin sorghum | | 25% low tannin sorghum | |
| | AMG-NL | JPO-172 | AMG NL | JPO-172 |
| Glucose | 173.0 ± 0.3 | 191.7 ± 2.2 | 153.6 ± 2.1 | 176.7 ± 0.8 |
| formation | | | | |
| [g/L] | | | | |
| Relative | 100.0 | 110.8 ± 1.1 | 100.0 | 115 ± 1.1 |
| glucose | | | | |
| yield [%] | | | | |

TABLE 7
Comparison of glucose concentration in wort produced
by AMG NL or AMG JPO-172, respectively using two
sorghum grist compositions and regime B for mashing

| | Mashing regime B | | | |
|---|---|---|---|---|
| | | | 75% high tannin + | |
| | 100% low tannin sorghum | | 25% low tannin sorghum | |
| | AMG NL | AMG JPO-172 | AMG NL | AMG JPO-172 |
| Glucose | 142.7 ± 0.8 | 203.3 ± 4.2 | 132.1 ± 2.7 | 189.7 ± 3.9 |
| formation | | | | |
| [g/L] | | | | |
| Relative | 100.0 | 142.5 ± 2.2 | 100.0 | 143.6 ± 0.1 |
| glucose | | | | |
| yield [%] | | | | |

TABLE 8
Impact of mashing regimes A and B on glucose concentration in wort produced
from low tannin sorghum with AMG NL or AMG JPO-172, respectively

| | 100% low tannin sorghum | | | |
|---|---|---|---|---|
| | AMG NL | | AMG JPO-172 | |
| | A | B | A | B |
| Glucose formation [g/L] | 173.0 ± 0.3 | 142.7 ± 0.8 | 191.7 ± 2.2 | 203.3 ± 4.2 |
| Relative glucose yield [%] | 100.0 | 82.5 ± 0.3 | 100.0 | 106.1 ± 0.1 |
| Extract yield [% of dry | 80.2 ± 0.2 | 82.7 ± 0.2 | 79.7 ± 0.0 | 83.5 ± 0.4 |
| matter grist] | | | | |

TABLE 9
Impact of mashing regimes A and B on glucose concentration
in wort produced from 75% high and 25% low tannin
sorghum with AMG NL or AMG JPO-172, respectively

| | 75% high tannin + 25% low tannin sorghum | | | |
|---|---|---|---|---|
| | AMG NL | | AMG JPO-172 | |
| | A | B | A | B |
| Glucose formation [g/L] | 153.6 ± 2.1 | 132.1 ± 2.7 | 176.7 ± 0.8 | 189.7 ± 3.9 |
| Relative glucose yield [%] | 100.0 | 86.0 ± 0.6 | 100.0 | 107.4 ± 1.7 |
| Extract yield [% of dry | 73.2 ± 0.1 | 78.5 ± 0.1 | 73.5 ± 0.1 | 78.8 ± 1.1 |
| matter grist] | | | | |

### Example 8. Mashing Experiment with 45% Malt and 55% Broken Rice

TABLE 10
Moisture of malt and rice used in trials

| | Moisture [%] |
|---|---|
| Barley malt | 7.6 |
| Broken rice | 12.2 |

Barley malt and broken rice was milled using a Lab mill 3100 (Perkin Elmer, Sweden) and retention sieve 0.8 mm (mm nominal diameter).

Mashing water was prepared by adding CaCl₂) to de-ionized water to achieve a final concentration of 100 ppm Ca²⁺-ions. Enzyme dilutions were prepared in mashing water targeting the addition of 0.2 g enzyme dilution to 17.8 g mashing water. 3.3 g rice and 2.7 g malt grist were added to RVA aluminium cans and manually mixed with 17.8 g pre-warmed (55° C.) mashing water, containing calcium-ions and enzymes according to Table 11 below, and immediately placed in a Rapid Visco Analyzer RVA 4500 (Perkin Elmer, Sweden).

TABLE 11
Enzyme dosages applied at the beginning of mashing

| | | EC- | Dosage [mg EP/kg grist] | |
|---|---|---|---|---|
| | Enzyme | number | Reference | Test |
| SEQ ID NO: 2 | AMG NL | 3.2.1.3 | 171.4 | — |
| SEQ ID NO: 6 | JPO-172 | 3.2.1.3 | — | 171.4 |
| SEQ ID NO: 12 | Alpha-amylase | 3.2.1.1 | 2.79 | 2.79 |

Mashing was conducted according to the following mashing regimes:
Mashing regime A: 20 minutes rest at 55° C.; heating from 55 to 78° C., with a heating rate of 1° C. per minute; 127 minutes rest at 78° C.; cooling to room temperature within 6 minutes. Total time without cooling=170 minutes.
Mashing regime B: 20 minutes rest at 55° C.; heating from 55 to 80° C., with a heating rate of 1° C. per minute; 125 minutes rest at 80° C.; cooling to room temperature within 6 minutes. Total time without cooling=170 minutes.
Mashing regime C: 20 minutes rest at 55° C.; heating from 55 to 85° C., with a heating rate of 1° C. per minute; 120 minutes rest at 85° C.; cooling to room temperature within 6 minutes. Total time without cooling=170 minutes.
Mashing regime D: 20 minutes rest at 55° C.; heating from 55 to 87° C., with a heating rate of 1° C. per minute; 118 minutes rest at 87° C.; cooling to room temperature within 6 minutes. Total time without cooling=170 minutes.

After mashing all mashes were adjusted for evaporation losses to the original weight of 24.0 g at start mashing. The mashes were then centrifuged for 10 min at 4000 rpm at 8° C. with a Heraeus 3 S-R benchtop centrifuge (Heraeus, Germany) and the supernatant immediately frozen and stored until subsequent analyses.

### Analyses:

Moisture of finely ground malt and rice grist, obtained from milling with laboratory hammermill Lab mill 3100 (Perkin Elmer, Sweden) with retention sieve 0.8 mm (mm nominal diameter), was determined using a HR73 Halogen Moisture Analyzer (Mettler Toledo, Switzerland) set to drying temperature of 105° C.

Glucose in wort was analysed with a Dionex HPLC system ICS-5000 using a RI-101 Differential Refractive Index Detector (Thermo Scientific, USA), based on Analytica-EBC method 8.7-Fermentable carbohydrates in wort by HPLC.

### Results:

Mashing trials conducted with 45% barley malt plus 55% broken rice confirmed the observations made in the previous sorghum mashing example. In this experiment the mashing temperature for simultaneous liquefaction and saccharification was tested between 78-87° C. (maximal temperature in sorghum mashing was 85° C.) and within this temperature range a continues improvement with increasing temperature could be observed for combinations of the thermostabilized glucoamylase JPO-172 with thermostable alpha-amylase.

The formation of glucose by AMG JPO-172 was constantly increasing with increasing final mashing temperature from 189.1 to 206.6 g/L, whereas concentrations obtained in worts in which AMG NL was applied significantly decreased from 184.7 to 116.4 g/L when the final mashing temperature was increased from 78 to 85° C. (see Tables 12 and 13), resulting in delta differences ranging from 2.4 to 76.3%. Increasing the final mashing temperature to 87° C. did not result in a further decrease in glucose concentration, indicating that the glucose was already formed in the heating phase from prior to the final temperature of 87° C.

TABLE 12
Comparison of glucose concentration in wort produced by
AMG NL or AMG JPO-172, respectively using 45% malt and
55% broken rice in four different mashing regimes A-D.

| | A | | B | | C | | D | |
|---|---|---|---|---|---|---|---|---|
| | | AMG | | AMG | | AMG | | AMG |
| | AMG | JPO- | AMG | JPO- | AMG | JPO- | AMG | JPO- |
| | NL | 172 | NL | 172 | NL | 172 | NL | 172 |
| Glucose | 184.7 | 189.1 | 168.3 | 194.1 | 116.4 | 200.2 | 117.2 | 206.6 |
| formation | | | | | | | | |
| [g/L] | | | | | | | | |
| Relative | 100.0 | 102.4 | 100.0 | 115.4 | 100.0 | 172.0 | 100.0 | 176.3 |
| glucose | | | | | | | | |
| yield [%] | | | | | | | | |

TABLE 13
Impact of mashing regimes A-D on glucose concentration
in wort produced from 45% malt and 55% broken rice
with AMG NL or AMG JPO-172, respectively

| | AMG NL | | | | AMG JPO-172 | | | |
|---|---|---|---|---|---|---|---|---|
| | A | B | C | D | A | B | C | D |
| Glucose | 184.7 | 168.3 | 116.4 | 117.2 | 189.1 | 194.1 | 200.2 | 206.6 |
| formation | | | | | | | | |
| [g/L] | | | | | | | | |
| Relative | 100.0 | 91.1 | 63.0 | 63.4 | 100.0 | 102.7 | 105.9 | 109.3 |
| glucose | | | | | | | | |
| yield [%] | | | | | | | | |

### Example 9. Mashing Experiment with 45% Malt and 55% Broken Rice and Other Thermostable JPO Variants

TABLE 14
Moisture of malt and rice used in trials

| | Moisture (%) |
|---|---|
| Barley malt | 7.6 |
| Broken rice | 12.2 |

Barley malt and broken rice was milled using a Lab mill 3100 (Perkin Elmer, Sweden) and retention sieve 0.8 mm (mm nominal diameter).

Mashing water was prepared by adding CaCl₂) to de-ionized water to achieve a final concentration of 100 ppm Ca²⁺-ions.

Enzyme dilutions were prepared in mashing water targeting the addition of 0.2 g enzyme dilution to 17.8 g mashing water. 3.3 g rice and 2.7 g malt grist were added to RVA aluminium cans and manually mixed with 17.8 g pre-warmed (55° C.) mashing water, containing calcium-ions and enzymes, and immediately placed in a Rapid Visco Analyzer RVA 4500 (Perkin Elmer, Sweden).

TABLE 15
Enzyme dosages applied at the beginning of mashing

| | | | | 45% barley malt + | |
|---|---|---|---|---|---|
| | | | | 55% broken rice | |
| | | Melting temperature | | Dosage [mg EP/kg grist] | |
| Sequence | Enzyme | at pH 5 or 4.3* [° C.] | EC-number | Reference | Test |
| SEQ ID NO: 2 | AMG NL | 83.4* | 3.2.1.3 | 171.4 | — |
| SEQ ID NO: 6 | AMG JPO-172 | 89.8 | 3.2.1.3 | — | 171.4 |
| See table 2 | AMG JPO-048 | 84.2 | 3.2.1.3 | — | 171.4 |
| See table 2 | AMG JPO-081 | 85.4 | 3.2.1.3 | — | 171.4 |
| See table 2 | AMG JPO169 | 89.5 | 3.2.1.3 | — | 171.4 |
| See table 2 | AMG JPO168 | 89.2 | 3.2.1.3 | — | 171.4 |
| SEQ ID NO: 12 | Amylolytic enzyme | n.d. | 3.2.1.1 | 2.79 | 2.79 |

Mashing was consequently conducted according to the following mashing regimes:
Mashing regime A: 20 minutes rest at 55° C.; heating from 55 to 78° C., with a heating rate of 1° C. per minute; 127 minutes rest at 78° C.; cooling to room temperature within 6 minutes. Total time without cooling=170 minutes.
Mashing regime D: 20 minutes rest at 55° C.; heating from 55 to 87° C., with a heating rate of 1° C. per minute; 118 minutes rest at 87° C.; cooling to room temperature within 6 minutes. Total time without cooling=170 minutes.

After mashing all mashes were adjusted for evaporation losses to the original weight of 24.0 g at start mashing. The mashes were then centrifuged for 10 min at 4000 rpm at 8° C. with a Heraeus 3 S-R benchtop centrifuge (Heraeus, Germany) and the supernatant immediately frozen and stored until subsequent analyses.

### Analyses:

Moisture of finely ground malt and rice grist, obtained from milling with laboratory hammermill Lab mill 3100 (Perkin Elmer, Sweden) with retention sieve 0.8 mm (mm nominal diameter), was determined using a HR73 Halogen Moisture Analyzer (Mettler Toledo, Switzerland) set to drying temperature of 105° C.

Glucose in wort was analysed with a Dionex HPLC system ICS-5000 using a RI-101 Differential Refractive Index Detector (Thermo Scientific, USA), based on Analytica-EBC method 8.7-Fermentable carbohydrates in wort by HPLC.

### Results:

The formation of glucose by different glucoamylase variants having a melting point of >87° C.: JPO168, JPO169 or JPO-172, increased by 7.4 to 9.3% when the main rest in mashing was at held at 87° C. compared to 78° C.

On the other hand, a decrease in glucose formation of 19.6% to 36.6% was observed for the variants having a melting point of <=85° C.: JPO048 or JPO081, when increasing the main rest from 78 to 87° C. (see Table 15 below).

These observations support that thermostable glucoamylase variants according to the instant invention, which can act at temperatures, where sufficient gelatinization and liquefaction by thermostable alpha-amylase is obtained, i.e in the range of at 87° C. will enable an efficient and simple mashing process.

TABLE 16
Comparison of glucose concentration in wort produced
by amyloglucosidase variants with different
thermo-stability, using 45% malt and 55% broken
rice in two different mashing regimes.

| | | Glucose | Relative |
|---|---|---|---|
| | | formation | glucose |
| | Mashing regime | [g/L] | yield [%] |
| AMG NL | A | 184.7 | 100.0 |
| | D | 117.2 | 63.4 |
| AMG JPO-172 | A | 189.1 | 100.0 |
| | D | 206.6 | 109.3 |
| AMG JPO048 | A | 191.2 | 100.0 |
| | D | 153.7 | 80.4 |
| AMG JPO081 | A | 193.2 | 100.0 |
| | D | 184.1 | 95.3 |
| AMG JPO169 | A | 197.2 | 100.0 |
| | D | 211.9 | 107.4 |
| AMG JPO168 | A | 189.5 | 100.0 |
| | D | 203.9 | 107.6 |

### Example 10. Mashing Experiment with 100% Malt

TABLE 17
Moisture of malt used in trials

| | Moisture (%) |
|---|---|
| Barley malt | 6.1 |

Malt was milled using a laboratory disk mill DLFU (Bühler AG, Switzerland) with a gap distance of 1.3 mm and the obtained grist stored dry in a closed container until further use.

Mashing water was prepared by adding CaCl2 to de-ionized water to achieve a final concentration of 100 ppm Ca2+-ions. The mashing water was distributed to mashing beakers in portions of 192 g (corresponding to a water:grist ratio of 3:1) and placed in a LP Electronic Mashing Device (Lochner GmbH, Germany) and subsequently pre-warmed to mash-in temperature of 55° C.

Malt grist was added to the mashing water at portions of 64 gram per beaker. The water and grist were homogenized by stirring for 5 minutes at 150 rpm. pH was adjusted to 5.2 by using lactic acid (conc. 20%). Stirring speed was subsequently reduced to 100 ppm and after this step, enzymes were added according to Table 18 below, and the mashing process started. The following enzymes were added:
Attenuzyme® Pro (Novozymes A/S) is a commercial product for so-called high attenuation in brewing comprising a glucoamylase from Talaromyces/Rasamsonia emersonii as well as a pullulanase (PulC) from Bacillus acidopullulyticus.
Attenuzyme® Core (Novozymes A/S) is a commercial product for so-called high attenuation in brewing comprising a glucoamylase (AMG-T) from Talaromyces/Rasamsonia emersonii.
Diazyme® 87 (International Flavors and Fragrances Inc., IFF) is a commercial product declared as an efficient blend of glucoamylases to maximize starch conversion, to be added at saccharification at temperatures below 66° C. according to the manufacturer.

TABLE 18
Enzyme dosages applied at the beginning
of 100% malt mashing, at 52° C.

| | | Dosage | Dosage |
|---|---|---|---|
| | | [AMG mg | [Pullulanase mg |
| Sequence | Enzyme | EP/kg grist] | EP/kg grist] |
| Commercial | Attenuzyme ® Pro | 81.25 | 1.47 |
| Commercial | Attenuzyme ® Core | 81.25 | — |
| Commercial | Diazyme ® 87 | 81.25 | — |
| SEQ ID NO: 6 | JPO-172 | 81.25 | — |

Mashing was conducted according to the following mashing regimes:
Mashing regime A: 20 minutes rest at 52° C.; heating from 52 to 64° C., with a heating rate of 1° C. per minute; 60 minutes rest at 64° C.; heating from 64 to 72° C., with a heating rate of 1° C. per minute; 15 minutes rest at 72° C.; heating from 72 to 78° C., with a heating rate of 1° C. per minute; 30 minutes rest at 78° C. Total time until mashing-off at 78° C.=151 minutes. After mashing, all mashes were adjusted for evaporation losses to the original weight of 256 g at start mashing.

At the end of mashing regime A, the whole sample is transferred to 600 ml centrifuge cups and centrifuged for 5 minutes at 4600 rpm (4566 g) in Heraeus Multifuge 3 S-R tabletop centrifuge equipped with Sorvall 75006445 rotor, to separate the sweet wort from the grist. 100 grams of sweet wort are decanted into 50 grams of mashing water (preheated to 78° C.) and the mashing process B started.
Mashing regime B: 30 minutes rest at 78° C.; heating from 78 to 96° C., with a heating rate of 0.6° C. per minute; cooling to room temperature within 10 minutes. Total time without cooling=60 minutes.

After mashing all mashes were adjusted for evaporation losses to the original weight of 150 g at start of mashing regime B. The mashes were then filtered at room temperature using filter-funnels equipped with MN614 Ø 240 mm (Macherey-Nagel, Germany) filter-paper. The obtained wort was immediately frozen and stored until subsequent analyses.
Mashing regime C: 20 minutes rest at 52° C.; heating from 52 to 64° C., with a heating rate of 1° C. per minute; 15 minutes rest at 64° C.; heating from 64 to 72° C., with a heating rate of 1° C. per minute; 15 minutes rest at 72° C.; heating from 72 to 78° C., with a heating rate of 1° C. per minute; 30 minutes rest at 78° C. Total time until mashing-off at 78° C.=106 minutes. After mashing, all mashes were adjusted for evaporation losses to the original weight of 256 g at start mashing.

At the end of mashing regime C, the whole sample is transferred to 600 ml centrifuge cups and centrifuged for 5 minutes at 4600 rpm (4566 g) in Heraeus Multifuge 3 S-R tabletop centrifuge equipped with Sorvall 75006445 rotor, to separate the sweet wort from the grist. 100 grams of sweet wort are decanted into 50 grams of mashing water (preheated to 78° C.) and the mashing process B started.

### Analyses:

Moisture of malt grist, obtained from milling with a laboratory disk mill DLFU (Bühler AG, Switzerland) with a gap distance of 1.3 mm, was determined using a HR73 Halogen Moisture Analyzer (Mettler Toledo, Switzerland) set to drying temperature of 105° C.

Glucose in wort was analysed with a Dionex HPLC system ICS-5000 using a RI-101 Differential Refractive Index Detector (Thermo Scientific, USA), based on Analytica-EBC method 8.7-Fermentable carbohydrates in wort by HPLC.

### Results:

Mashing with glucoamylase JPO-172 (SEQ ID NO: 6) resulted in significantly higher final glucose concentrations than that achieved with the three commercial glucoamylases tested: Attenuzyme® Pro, Attenuzyme® Core and Diazyme® 87; see Tables 19 and 20.

When comparing the composition of the sweet wort produced in a 151 minutes mashing regime (A), wort produced with AMG JPO-172 (SEQ ID NO: 6) resulted in 146.4% higher final glucose concentration.

Following the observation that the thermostable amyloglucosidase JPO-172 (SEQ ID NO: 6) can release higher level of glucose than the tested commercial products in a mashing regime where saccharification is extended for 60 minutes at 64° C. followed by 15 minutes rest at 72° C., a shorter mashing program was applied.

A shorter mashing regime, C, was designed to have 50% shorter saccharification between 64° C. and 78° C. (44.5 minutes compared to 89 minutes), reached by decreasing the saccharification step at 64° C. to 15 minutes. The resulting overall process was 22% shorter (166 minutes compared to 211 minutes). The thermostable amyloglucosidase JPO-172 was tested in this mashing regime (Table 19). Wort produced with AMG JPO-172 (SEQ ID NO: 6) applying the shorter saccharification mashing regime C resulted in a final glucose concentration that was higher than what was previously observed with the tested commercial enzymes in a longer mashing. Relatively to Attenuzyme® Pro in the 45 minutes longer mashing, used as benchmark, the glucose yield obtained in wort produced with JPO-172 in the shorter process was 123.6% (Table 19).

TABLE 19
Comparison of glucose concentration in wort produced
by commercial products containing amyloglucosidase
variants with different thermostabilities using
100% malt in different mashing regimes.

| | | | Relative |
|---|---|---|---|
| | Mashing | Glucose | glucose |
| | Regime | formation [g/L] | yield [%] |
| Attenuzyme ® Pro | A - 151 minutes | 78.53 | 100.0 |
| Attenuzyme ® Core | A - 151 minutes | 77.82 | 99.1 |
| Diazyme ® 87 | A - 151 minutes | 49.00 | 62.4 |
| JPO-172 | A - 151 minutes | 114.98 | 146.4 |
| JPO-172 | C - 106 minutes | 97.11* | 123.6 |

*Determined in quadruplicate - average value in table.

After mashing-off at the end of regime A and C, sweet wort is collected and mixed with sparging water (in 2:1 ratio) to simulate sparging and transferred to mashing regime B, for a additional 60 minutes' process.

Relatively to Attenuzyme Pro®, used as benchmark, the glucose yield obtained in cold wort produced with JPO-172 in the 211 minutes process (mashing A+B) was 176.7% (Table 20).

Wort produced with AMG JPO-172 (SEQ ID NO: 6) applying a shorter saccharification mashing regime (C+B) resulted in a final glucose concentration that was higher than what previously observed with other commercial enzymes in a longer mashing. Relatively to Attenuzyme Pro® in 45 minutes longer mashing, used as benchmark, the glucose yield obtained in cold wort produced with AMG JPO-172 in the 166 minutes process (mashing C+B) was 160.5% (Table 20).

These observations support that thermostable glucoamylase JPO-172 (SEQ ID NO: 6), which can act at temperatures higher than 78° C., will enable an efficient and simple mashing process with shorter saccharification to achieve higher fermentable sugar and higher wort composition in glucose.

TABLE 20
Comparison of glucose concentration in wort produced by commercial
products containing amyloglucosidase variants with different
thermostability using 100% malt in different mashing regimes.

| | | Glucose | Relative |
|---|---|---|---|
| | Mashing | formation | glucose |
| | Regime | [g/L] | yield [%] |
| Attenuzyme Pro ® | A + B | 49.35 | 100.0 |
| | 211 minutes | | |
| Attenuzyme Core ® | A + B | 48.72 | 98.7 |
| | 211 minutes | | |
| Diazyme 87 ® | A + B | 31.58 | 64.0 |
| | 211 minutes | | |
| AMG JPO-172 | A + B | 87.21 | 176.7 |
| | 211 minutes | | |
| AMG JPO-172 | C + B | 79.21 | 160.5 |
| | 166 minutes* | | |

*Determined in quadruplicate - average value in table.

1. A method of producing a brewer's wort comprising adding to a mash a mature thermostable variant of a parent glucoamylase with at least 70% sequence identity to SEQ ID NO: 1, SEQ ID NO: 6, SEQ ID NO: 7, SEQ ID NO: 8 or SEQ ID NO: 10 before, during or after liquefaction of starch.

2. The method according to claim 1, wherein the parent glucoamylase is from a species of Penicillium, preferably from Penicillium oxicalum, Penicillium miczynskii, Penicillium russellii or Penicillium glabrum.

3. The method according to claim 1, wherein the mature variant comprises at least one amino acid modification in one or more or all of the positions corresponding to positions 1, 2, 4, 6, 7, 11, 31, 34, 50, 65, 79, 103, 132, 327, 445, 447, 481, 484, 501, 539, 566, 568, 594 and 595 in SEQ ID NO: 1.

4. The method according to claim 3, wherein the at least one amino acid modification comprises a substitution in one or more or all of the positions corresponding to positions 1, 2, 4, 11, 65, 79 and 327 in SEQ ID NO: 1.

5. The method according to claim 3, wherein the at least one amino acid modification comprises a substitution in one or more or all of the positions corresponding to positions 1, 6, 7, 31, 34, 79, 103, 132, 445, 447, 481, 566, 568, 594 and 595 in SEQ ID NO: 1.

6. The method according to claim 3, wherein the at least one amino acid modification comprises a substitution in one or more or all of the positions corresponding to positions 1, 6, 7, 31, 34, 50, 103, 132, 445, 447, 481, 501, 539, 566, 568, 594 and 595 in SEQ ID NO: 1.

7. The method according to claim 3, wherein the at least one amino acid modification comprises a substitution in one or more or all of the positions corresponding to positions 1, 6, 7, 31, 34, 50, 103, 132, 445, 447, 481, 501, 539, 566, 568, 594 and 595 in SEQ ID NO: 1.

8. The method according to claim 3, wherein the at least one amino acid modification comprises a substitution in one or more or all of the positions corresponding to positions 1, 6, 7, 31, 34, 50, 79, 103, 132, 445, 447, 481, 484, 501, 539, 566, 568, 594 and 595 in SEQ ID NO: 1.

9. The method according to claim 1, wherein the mature thermostable variant has a thermostability improvement (Td) over its parent of at least 5° C.

10. The method according to claim 1, wherein the mature thermostable variant has a relative activity at 91° C. of at least 150 compared to its parent.

11. The method according to claim 1, wherein the mashing comprises an incubation step at a temperature of 65° C. or higher for at least 20 minutes.

12. The method according to claim 1, wherein the pH of the mash is about 4.6 to about 6.4.

13. The method according to claim 1, wherein a target glucose concentration is reached in a shorter saccharification time compared to when a less thermostable AMG enzyme than that of claim 1, has been added.

14. The method according claim 1, comprising also adding one or more additional enzyme to the mash, said additional enzyme selected from the group consisting of a alpha amylase, maltogenic amylase, raw-starch degrading alpha amylase, beta amylase, aminopeptidase, carboxypeptidase, catalase, cellobiose oxidase, cellulase, chitinase, cutinase, cyclodextrin glycosyltransferase, deoxyribonuclease, esterase, glucan 1,4-alpha-maltotetrahydrolase, glucanase, beta glucanase, galactanase, alpha-galactosidase, beta-galactosidase, glucose oxidase, alpha-glucosidase, beta-glucosidase, haloperoxidase, hemicellulytic enzyme, invertase, laccase, lipase, mannanase, mannosidase, oxidase, pectinolytic enzymes, peptidoglutaminase, peroxidase, phospholipase, phytase, polyphenoloxidase, protease, pullulanase, ribonuclease, transglutaminase, and xylanase.

15. The method according to claim 1, wherein the wort has more than 80% glucose, compared to the total carbohydrate content of the wort.

16. A mashing or brewing composition comprising a mature thermostable variant of a parent glucoamylase as defined in claim 1.

17. The mashing or brewing composition of claim 16, which also comprises one or more additional enzyme selected from the group consisting of a alpha amylase, maltogenic amylase, raw-starch degrading alpha amylase, beta amylase, aminopeptidase, carboxypeptidase, catalase, cellobiose oxidase, cellulase, chitinase, cutinase, cyclodextrin glycosyltransferase, deoxyribonuclease, esterase, glucan 1,4-alpha-maltotetrahydrolase, glucanase, beta glucanase, galactanase, alpha-galactosidase, beta-galactosidase, glucose oxidase, alpha-glucosidase, beta-glucosidase, haloperoxidase, hemicellulytic enzyme, invertase, laccase, lipase, mannanase, mannosidase, oxidase, pectinolytic enzymes, peptidoglutaminase, peroxidase, phospholipase, phytase, polyphenoloxidase, protease, pullulanase, ribonuclease, transglutaminase, and xylanase.

18. The method according to claim 3, wherein the at least one amino acid modification comprises a substitution in one or more or all of the positions corresponding to R1A, P2N, P4S, P11F, T65A, K79V and Q327F in SEQ ID NO: 1.

19. The method according to claim 3, wherein the at least one amino acid modification comprises a substitution in one or more or all of the positions corresponding to R1A, G6S, G7T, R31F, K34Y, E50R, K79V, S103N, A132P, D445N, V447S, S481P, T484P, E501A, N539P, D566T, T568V, Q594R and F595S in SEQ ID NO: 1.
